(19) United States
(12) Patent Application Publication
(10) Pub. No.: US 2024/0199278 A1
(43) Pub. Date: Jun. 20, 2024

(54) Spring Carrier

(71) Applicant: Sanofi Winthrop Industrie, Gentilly (FR)

(72) Inventors: Philippe Chabert, Paris (FR); David Darras, Paris (FR)

(21) Appl. No.: 18/572,439

(22) PCT Filed: Jun. 23, 2023
(86) PCT No.: PCT/EP2022/067086

§ 371 Date: Dec. 20, 2023

(30) Foreign Application Priority Data
Jun. 24, 2021 (EP) 21315104.6

(51) Int. Cl. B65D 25/10; B65D 25/54; B65D 85/02

(52) U.S. Cl. CPC B65D 25/101; B65D 25/54; B65D 85/02

(57) ABSTRACT

A spring carrier for receiving, retaining and discharging of a coil spring in a manufacturing assembly process comprising an elongate hollow body defining an inner cavity configured to receive a coil spring and an opening at a first proximal end of the hollow body for the insertion and/or extraction of a coil spring into/from the inner cavity. The hollow body includes a second distal end opposite to the first proximal end. At least one deflectable member is located proximate to the second distal end of the hollow body and includes a retaining formation which is configured to engage a coil spring when located within the inner cavity. The deflectable member is movable between a first unbiased position, whereby the retaining formation extends into the inner cavity to engage the coil spring when located within the inner cavity, and a second biased position, whereby the retaining formation is disposed outwardly to disengage a coil spring when located within the inner cavity. An apparatus comprising such a spring carrier, and a method of manipulating a coil spring using such a spring carrier, are also disclosed.

### FIELD OF THE INVENTION

The present invention relates to a device for carrying a spring, an apparatus including such a device, and a method of use of such a device and apparatus.

### BACKGROUND

Many devices require one or more springs and the method and apparatus for assembly of such devices requires accurate and repeated retrieval, movement and placement of such springs. Devices including one or more springs in their assembly include medicament injection devices. Such devices may include a spring to facilitate various functions of the device, including operation of a drug administration mechanism, or deployment of one or more safety features before, during or after a medicament delivery process.

Springs can readily become entangled if stored or conveyed together in a bulk manner, and separating springs when required to be assembled into a device being manufactured can be difficult and time consuming, and therefore inefficient and costly in terms of the manufacturing process. In high-volume manufacturing processes, errors in an assembly line or a need to pause a production line, for example due to a jam or fault in the machinery, is undesirable as it leads to lost production time, loss of productivity and product output, and impacts manufacturing and product costs.

In a manufacturing process of a product containing one or more springs, it is therefore desirable to provide a device that facilitates repeated and reliable retrieval, transport, and placement of such springs for use in such a process, and/or which may help protect and ensure spring integrity.

### SUMMARY

According to the present disclosure, there is provided a spring carrier for receiving, retaining and discharging of a coil spring in a manufacturing assembly process comprising an elongate hollow body defining an inner cavity configured to receive a coil spring, an opening at a first proximal end of the hollow body for the insertion and/or extraction of a coil spring into/from the inner cavity, the hollow body including a second distal end opposite to the first proximal end, at least one deflectable member located proximate to the second distal end of the hollow body and including a retaining formation which is configured to engage and retain a coil spring when located within the inner cavity, wherein the deflectable member is movable between a first unbiased position, whereby the retaining formation extends into the inner cavity to engage and retain the coil spring when located within the inner cavity, and a second biased position whereby the retaining formation is disposed outwardly to disengage a coil spring when located within the inner cavity.

The retaining formation may extend further outwardly when the deflectable member is in the second position than when the deflectable member is in the first position. The retaining formation being disposed outwardly in the second biased position may comprise being outwardly relative to a central axis or a surface of a side wall of the spring carrier, and may be radially outwardly thereof.

The retaining formation may be disposed outwardly of an inner surface of the inner cavity when the deflectable member is in the second biased position.

The deflectable member may extend substantially parallel to a central axis of the hollow body in the first position.

The deflectable member may be in a relaxed state in the first unbiased position and may be elastically deformed in the second biased position The deflectable member may include an actuation feature for engagement with an actuator to deflect the deflectable member from the first position to the second position.

The actuation feature may comprise a contact surface disposed at an acute angle with respect to the central axis of the hollow body.

The actuation feature may comprise a head located distally on the deflectable member, and the head may include the contact surface which may comprise a ramp disposed at an acute angle with respect to the central axis of the hollow body.

The deflectable member may include an abutment step for engagement with an end of a coil spring when located within the inner cavity. The abutment step may comprise a surface facing the first proximal end. The abutment step may lie in a plane substantially perpendicular to the central axis of the hollow body.

The retaining formation may comprise at least one projecting element extending inwardly from the deflectable member.

The retaining formation may comprise at least one notch configured to receive a portion of a coil spring when located within the inner cavity. The notch may be defined between the projecting element and the head.

The head may extend radially inwardly into the inner cavity by a greater distance than the projecting element.

The deflectable member may be integrally formed with a side wall of the hollow body.

The deflectable member may be disposed within an aperture in a side wall of the hollow body.

The hollow body may comprise a continuous annular portion extending entirely around the perimeter of the hollow body at a distal-most region of the second end and located further towards the second distal end than the deflectable member.

The deflectable member may comprise a resilient arm configured to flex about a fixed proximal end of the resilient arm.

The spring carrier may comprise a plurality of deflectable members. The plurality of deflectable members may be equally spaced around the perimeter of the hollow body. The spring carrier may comprise two deflectable members disposed diametrically opposite to each other on the hollow body.

The hollow body may be a cylindrical tube which is circular in cross-section. The hollow body may be substantially uniform in cross-section dimension along its length.

The hollow body may be substantially rigid and not readily deformable from its cross-sectional shape. The or each deflectable member may be deflectable between the first and second positions relative to a side wall of the hollow body.

The hollow body may include a flange extending radially outwardly from the hollow body. The flange may be located at the first proximal end of the hollow body.

The spring carrier may comprise an opening at the second distal end of the hollow body.

The opening at the second distal end of the hollow body may be of the same cross-sectional dimension as the cross-sectional dimension of the inner cavity.

The opening at the second distal end of the hollow body may be of a smaller cross-sectional dimension than the cross-sectional dimension of the inner cavity.

The spring carrier may comprise at least one window to allow a coil spring located within the spring carrier to be visible from outside the spring carrier through the window. The or each window may be formed in a side wall of the hollow body, and may be formed in a side wall of the hollow body in a location between the first proximate end and the second distal end of the hollow body. The or each window may be formed in the at least one deflectable member. The or each window may be formed in one or both of the side wall and the or each delectable member.

The opening at the first proximal end of the hollow body may comprise a tapered region such that the opening widens towards the first proximal end.

The opening at the second distal end of the hollow body may comprise a tapered region such that the opening widens towards the second distal end.

The second distal end of the hollow body may include one or more protrusions extending inwardly from the hollow body. The or each protrusion may extend at least partially across an opening at the second distal end of the hollow body. The second end of the hollow body may include an inwardly-protruding lip extending at least partially around an opening at the second distal end. The second distal end of the hollow body may be partially closed by an end wall.

The spring carrier may comprise one or more orientation features configured for cooperation with corresponding orientation features on an apparatus with which the spring carrier may be used. The orientation feature(s) may allow the spring carrier to be accurately aligned in use. Such orientation feature(s) may comprise one or more recesses or slots in the flange. Such orientation feature(s) may comprise diametrically opposed slots in the flange.

The projecting elements may be provided on a plurality of deflectable members. The or each projecting element on one deflectable member may be aligned in an axial direction of the hollow body with the or each corresponding projecting element on another deflectable member. The or each projecting element on one deflectable member may be off-set in an axial direction of the hollow body with the or each corresponding projecting element on another deflectable member

The projecting elements provided on the or each deflectable member may be of different sizes to each project by a different distance from the or each deflectable member. The projecting elements may increase in size and/or projecting distance in a direction towards a free end of the or each deflectable member, and/or in a direction towards the second distal end of the hollow body.

The or each deflectable member may be configured to deflect laterally outwardly in the second position by a distance of 1 mm-4 mm, and may be between 1 mm-3 mm, and may be between 1-2 mm, and may be around 1.5 mm.

The or each deflectable member may be configured to deflect laterally outwardly in the second position by an angle of around 4 to 12 degrees, and may be between 6 to 10 degrees, and may be around 8 degrees.

The or each deflectable member may comprise an angled restoring surface provided on an outer region of the resilient arm. The restoring surface may be angled inwardly in a direction towards the second, distal end of the hollow body.

The spring carrier may comprise one or more centering lugs projecting inwardly from an inside surface of a side wall of the hollow body. The centering lugs may project towards the central axis of the hollow body. The centering lugs may be equally spaced around the inside circumference of the side wall of the hollow body. The or each centering lug may be formed as ramp which increases in inward projecting distance in a direction towards the second distal end of the hollow body.

Also provided in the present disclosure is an apparatus comprising a spring carrier as described above, and an actuator configured for engagement with the deflectable member and operable to move the deflectable member from the first position to the second position.

The actuator may comprise an elongate rod configured to be inserted into an opening at the second distal end of the hollow body. The actuator may comprise any suitable material, including but not limited to plastic, metal, such as stainless steel, and magnetic material.

The actuator may comprise a chamfered or angled end configured to engage with the or each deflectable member.

The angle of the angled end or chamfer at the end of the actuator relative to a central axis of the actuator may be substantially equal to the angle of the contact surface of the deflectable member relative to a central axis of hollow body such that the angled end/chamfer and contact surface make surface contact when the actuator is engaged with the deflectable member.

The actuator may comprise an airflow passage extending through the actuator and configured for connection to an air source to generate a flow of air through the actuator and into the hollow body.

An air outlet may be provided in a distal end of the actuator and in fluid communication with the airflow passage to allow the flow of air through the actuator and out of the air outlet into the hollow body.

The air outlet is configured to direct the flow of air out of the air outlet at an acute angle other than parallel relative to the central axis of the actuator.

The actuator may include a narrowed section extending from the chamfered end and configured to be received within a coil spring when a coil spring is located within the spring carrier. The narrowed section of the actuator may be of a constant diameter along an axial length of the narrowed section. The narrowed section of the actuator may reduce in diameter along an axial length of the narrowed section in a direction towards a distal end of the actuator.

The actuator may include a magnetic portion configured to attract and retain a metallic coil spring thereto. Such magnetic portion may be provided on a remote end of the actuator which is inserted into the spring carrier in use. The magnetic portion may help in alignment and maintaining a desired position of a coil spring during an insertion step of the coil spring into the spring carrier.

The actuator may comprise at least one moveable jaw configured to be inserted into the spring carrier and to be moveable to engage and move the at least one deflectable member from the first unbiased position to the second biased position. The actuator may comprise a plurality of moveable jaws. The moveable jaws may be moveable away from each other to engage the or each deflectable member. The actuator may comprise a number of moveable jaws equal to the number of deflectable members provided on the spring carrier the actuator is configured to be used to actuate.

The or each moveable jaw may be moveable from a first disengaged position to a second, engaged position. The or each jaw may be arranged to form a rod in the first, disengaged position. The or each jaw may be moveable substantially radially outwardly from the rod-shaped disengaged position to the engaged position. The actuator may comprise a chuck to which the or each jaw may be moveably mounted.

Also provided in the present disclosure is a manufacturing apparatus comprising an apparatus described above, and a spring extraction station configured to receive the spring carrier and locate the spring carrier whilst the actuator is engaged with the spring carrier to allow extraction of the coil spring from the spring carrier.

Also provided in the present disclosure is an assembly system comprising an apparatus as described above, and a coil spring manufacturing machine, wherein the coil spring manufacturing machine is configured to produce a coil spring and the system further includes an insertion station arranged to feed the produced coil spring into the spring carrier.

The assembly system may further include the manufacturing apparatus comprising the extraction station described above.

Also provided in the present disclosure is a method of manipulating a coil spring using a spring carrier as described above, the method comprising moving the deflectable member from the first position to the second position, inserting the coil spring into the inner cavity through the opening at the first proximal end of the hollow body, and moving the deflectable member from the second position to the first position such that the retaining formation engages the coil spring to retain the coil spring within the inner cavity.

Also provided in the present disclosure is a method of manipulating a coil spring using a spring carrier as described above, the method comprising moving the deflectable member from the first position to the second position such that the retaining formation disengages a coil spring located within the inner cavity to allow the coil spring to be extracted from the inner cavity through the opening at the first proximal end of the hollow body.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a first proximal end of the hollow body, a second distal end opposite to the first proximal end, and at least one deflectable member located proximate to the second distal end of the hollow body and including a retaining formation, the method comprising moving the deflectable member from a first position whereby the retaining formation extends into the inner cavity, to a second position whereby the retaining formation extends outwardly, inserting the coil spring into the inner cavity through the opening at the first proximal end of the hollow body, and moving the deflectable member from the second position to the first position such that the retaining formation engages the coil spring to retain the coil spring within the inner cavity.

The method may comprise engaging an actuator with the or each deflectable member to move the deflectable member(s) from the first position to the second position, and disengaging the actuator after insertion of the coil spring into the inner cavity to allow the deflectable member(s) to move to the first position such that the retaining formation(s) engage(s) the coil spring to retain the coil spring within the inner cavity.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a first proximal end of the hollow body, a second distal end opposite to the first proximal end, and at least one deflectable member located proximate to the second distal end of the hollow body and including a retaining formation, the method comprising moving the deflectable member from a first position whereby the retaining formation extends into the inner cavity to a second position whereby the retaining formation extends outwardly such that the retaining formation disengages a coil spring located within the inner cavity to allow the coil spring to be extracted from the inner cavity through the opening at the first proximal end of the hollow body.

The method may comprise engaging an actuator with the or each deflectable member to move the deflectable member(s) from the first position to the second position such that the retaining formation(s) disengage(s) the coil spring located within the inner cavity.

Engaging the actuator with the or each deflectable member may comprise inserting the actuator into an opening at the second distal end of the hollow body.

The method may comprise moving the or each deflectable member to a different second position during a coil spring insertion process than a second position during a coil spring extraction process. The or each deflectable member may be deflected further outwardly in a coil spring extraction process than in a coil spring insertion process.

The spring carrier may comprise a window in at least one of a side wall of the hollow body and the at least one deflectable member, and the method may comprise detecting the presence or absence of a coil spring within the inner cavity of the hollow body by means of the or at least one of the windows. The detection of the presence or absence of a coil spring within the inner cavity of the hollow body by means of window(s) may comprise using a camera or optical sensor aligned with the window(s).

### BRIEF DESCRIPTION OF THE DRAWINGS

Embodiments will now be described, by way of example only, with reference to the accompanying drawings, in which:

FIG. 1 is a perspective view of a spring carrier of an embodiment of the present invention;

FIG. 2 is a cut-away perspective view of the spring carrier of FIG. 1;

FIG. 3 is an enlarged cut away perspective view of a region at a second end of the spring carrier of FIGS. 1 and 2;

FIG. 4 is an enlarged cross-sectional view of a region at the second end of the spring carrier of FIGS. 1 to 3;

FIGS. 5A-5E show a sequence of steps of use of the spring carrier of FIGS. 1-4 during insertion of a coil spring into the spring carrier;

FIGS. 6A-6E show a sequence of steps of use of the spring carrier of FIGS. 1-5E during extraction of a coil spring from the spring carrier;

FIG. 7A shows a sequence step similar to that of FIG. 5D but of a spring carrier of another embodiment of the invention;

FIG. 7B shows an enlarged view of a portion of FIG. 7A showing a deflectable member in a deflected state and retaining formations of the spring carrier, and an actuator;

FIG. 8A shows a sequence step similar to that of FIG. 5E but of the spring carrier of FIGS. 7A and 7B;

FIG. 8B shows an enlarged view of a portion of FIG. 8A showing a deflectable member in a relaxed state and retaining formations of the spring carrier, and coil spring secured within the spring carrier;

FIG. 9A shows a sequence step similar to that of FIG. 6D but of the spring carrier of FIGS. 7A to 8B;

FIG. 9B shows an enlarged view of a portion of FIG. 9A showing the deflectable member in a deflected state;

FIG. 10A shows a sequence step similar to that of FIG. 6E but of the spring carrier of FIGS. 7A to 9B;

FIG. 10B shows an enlarged view of a portion of FIG. 10A showing the deflectable member in a relaxed state;

FIG. 11 is a schematic view of an assembly system of an embodiment of the invention;

FIG. 12 is a schematic cross-sectional view of opposing deflectable members showing retaining formations of an embodiment of the invention;

FIG. 13 is a schematic cross-sectional view of opposing deflectable members showing retaining formations of another embodiment of the invention;

FIG. 14 is a schematic cross-sectional view of a deflectable member of an alternative embodiment to that of FIGS. 12 and 13 FIG. 15A is an enlarged view of a portion of a deflectable member of an embodiment of the invention showing a retaining formation of a first variant;

FIG. 15B is an enlarged view of a portion of a deflectable member of an embodiment of the invention showing a retaining formation of a second variant;

FIG. 15C is an enlarged view of a portion of a deflectable member of an embodiment of the invention showing a retaining formation of a third variant;

FIG. 16 is a side perspective view of a spring carrier of another embodiment of the invention;

FIG. 17A is a top perspective view of a spring carrier of another embodiment of the invention;

FIG. 17B is a bottom perspective view of the spring carrier of FIG. 17A;

FIG. 17C is a cross-sectional view of the spring carrier of FIGS. 17A and 17B;

FIG. 17D is an enlarged view of a portion of FIG. 17C although showing the deflectable members displaced slightly outwardly than in FIG. 17C, and a restoring tool disposed adjacent the spring carrier;

FIG. 17E shows an enlarged view similar to that of FIG. 17D but with the restoring tool engaging the deflectable members to restore them to be positioned flush with the outer surface of the spring carrier;

FIG. 18 is a bottom perspective view of a portion of a bottom end of a spring carrier of another embodiment of the invention;

FIG. 19A shows a sequence step similar to that of FIGS. 5D and 7A but of a spring carrier and actuator of another embodiment of the invention;

FIG. 19B shows an enlarged view of a portion of FIG. 19A showing a deflectable member in a deflected state and retaining formations of the spring carrier, and an actuator;

FIG. 20A shows a sequence step similar to that of FIGS. 5E and 8A but of the spring carrier of FIGS. 19A and 19B;

FIG. 20B shows an enlarged view of a portion of FIG. 20A showing a deflectable member in a relaxed state and retaining formations of the spring carrier, and coil spring secured within the spring carrier;

FIG. 21A is a perspective view of an actuator for use with the spring carrier of FIGS. 19A to 20B;

FIG. 21B is a cross-sectional view of the actuator of FIG. 21A;

FIG. 22 shows a partial cross-sectional view of a spring carrier of another embodiment of the invention;

FIG. 23 is a perspective end view of a spring carrier of another embodiment of the invention;

FIG. 24 is a cross-sectional view of the spring carrier of FIG. 23; and

FIGS. 25A-25E show a sequence of steps of use of a spring carrier of the invention with an alternative configuration of actuator.

### DETAILED DESCRIPTION

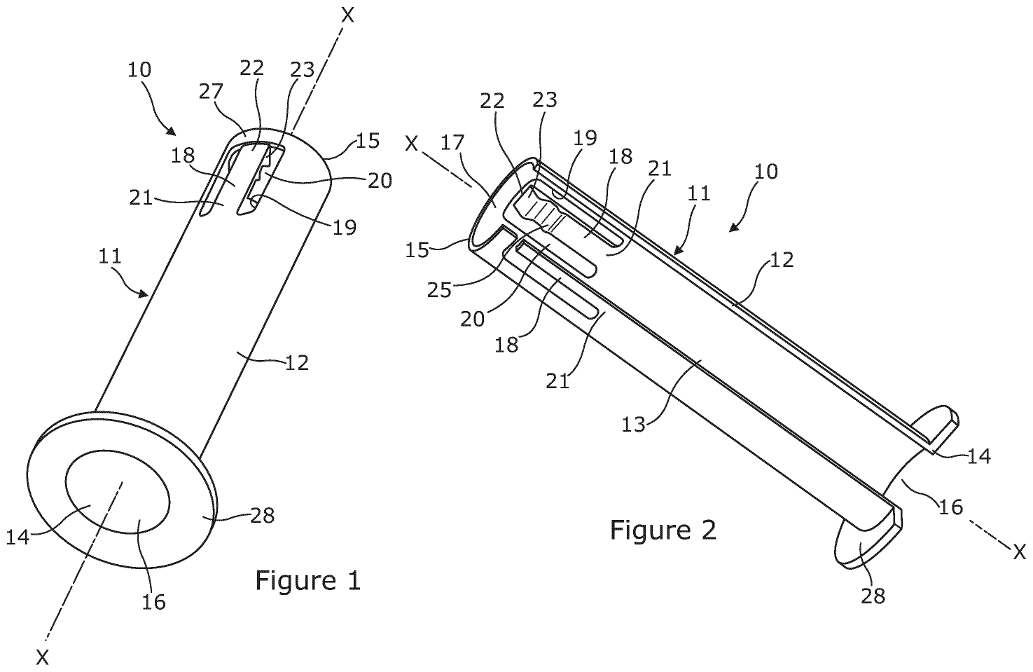

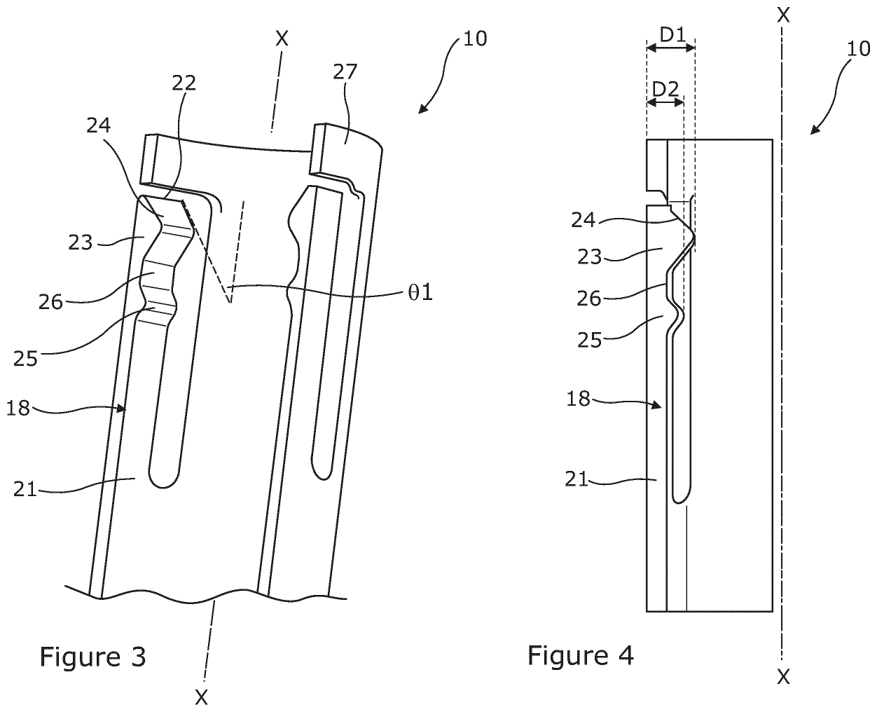

FIGS. 1 to 4 show a spring carrier 10 of an embodiment of the invention which comprises a hollow body 11 having a side wall 12 formed as a tube and defining an inner cavity 13. The hollow body 11 includes opposite first, proximal and second, distal ends 14, 15. The hollow body 11 is circular in cross-section and comprises a central axis X-X. A first opening 16 is provided at the first proximal end 14. In the exemplary embodiment, a second opening 17 is provided at the second distal end 15. The inner cavity 13 is accessible via both the first and second openings 16, 17.

The spring carrier 10 includes two deflectable members 18, which in the illustrated exemplary embodiment comprise resilient arms 18. The resilient arms 18 are provided in the side wall 12 of the hollow body 11. The resilient arms 18 are disposed within apertures 19 in the side wall 12 such that a space 20 is provided around the resilient arms 18. The resilient arms 18 are joined to the side wall 12 at a respective fixed end 21 thereof. The resilient arms 18 are configured to flex about the fixed end 21. The resilient arms 18 have a free end 22 at an opposite end of the respective resilient arm 18 to the fixed end 21. The resilient arms 18 include an actuation feature 23 for engagement with an actuator 30 (described in more detail below) operable to move the resilient arms 18 in use. In the exemplary embodiment shown, the actuation feature comprises a head 23 provided at the free end 22 of each resilient arm 18.

The head 23 comprises a contact surface 24 extending from the remote portion of the free end 22 of the respective resilient arm 18. The contact surface 24 comprises a ramped surface extending inwardly towards the central axis X-X and disposed at an acute angle θ1 with respect to the central axis X-X. The contact surface 24 may be configured at an angle θ1 of between 15 and 55 degrees with respect to the central axis X-X, and may be, for instance, between 20 and 50 degrees, and may be between 25 and 45 degrees, and may be between 30 and 40 degrees, and may be around 35 degrees.

The resilient arms 18 include retaining formations which are configured, in use, to engage with a coil spring C when a coil spring C is disposed within the inner cavity 13 and to retain the coil spring C in place within the inner cavity 13. The retaining formations comprise a projecting element 25 extending from the respective resilient arm 18, and directed inwardly towards the central axis X-X of the hollow body 11. The projecting elements 25 are spaced along the respective resilient arm 18 from the respective head 23 such that the retaining formations also comprise a notch 26 which is defined between the head 23 and projecting element 25 on each resilient arm 18.

The resilient arms 18 extend substantially parallel to the central axis X-X of the hollow body 11. The resilient arms 18 are moveable by being elastically deflected. The resilient arms 18 are in a relaxed state when in a first position, in which the resilient arms 18 extend substantially parallel to the central axis X-X of the hollow body 11, and substantially flush with the side wall 12 of the hollow body 11. The resilient arms 18 may be deflected away from the central axis X-X into a second position. The resilient arms are elastically deformed in the second position.

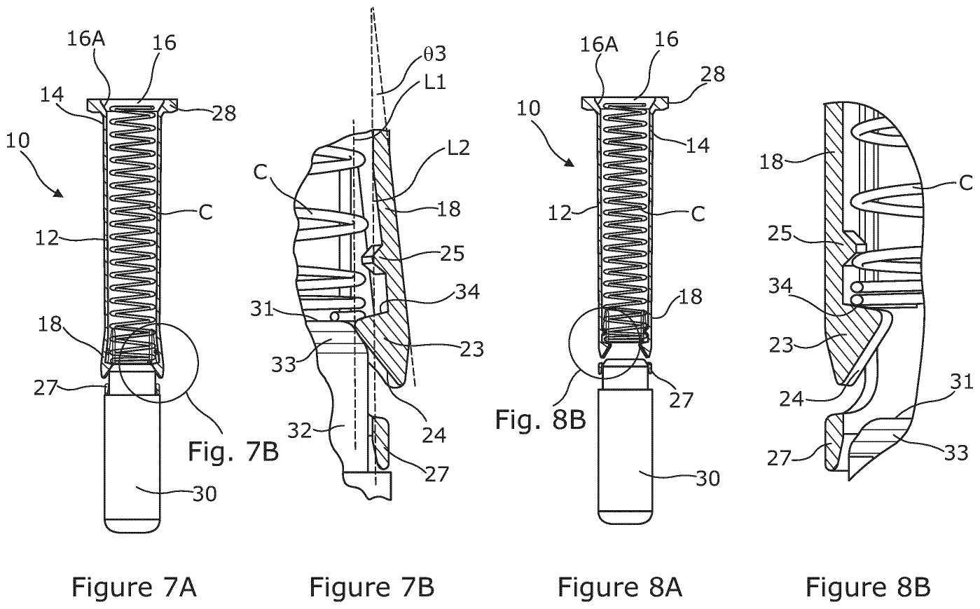

An inner-most portion of the projecting elements 25 may be disposed radially inwardly of the plane of the inner surface of the side wall 12 of the hollow body 11 when the respective resilient arm 18 is in the first, relaxed position. The head 23 may extend further inwardly towards the central axis X-X than the projecting element 25 of each resilient arm 18, at least in the first, relaxed position of the resilient arm 18. This can be seen, for example, in FIG. 4, in which a distance D1 between an outer surface of the hollow body 11 and an inner-most portion of the head 23, is greater than a distance D2 between the outer surface of the hollow body 11 and an inner-most portion of the projecting element 25. For instance, the head 23 extends further inwardly towards the central axis X-X than the projecting element 25 of each resilient arm 18 in both the first, relaxed position of the resilient arm 18 and also the second, elastically deformed position of the resilient arms 18. This configuration will permit a coil spring C to be insertable into the inner cavity 13 when the resilient arms 18 are in the second, deformed position and the projecting elements to be clear of the coil spring, but the coil spring C to be unable to pass beyond the head 23, even when the resilient arms 18 are in the second, deformed position. This will be explained in more detail below with reference to FIGS. 7A to 8B. However, the invention is not limited to this configuration and in other embodiments, the head 23 may extend an equal of lesser distance inwardly towards the central axis X-X than the projecting element 25 of each resilient arm 18, at least in the first, relaxed position of the resilient arm 18.

The hollow body 11 includes a continuous annular portion 27 at the distal-most region of the second end of the hollow body 11. The continuous annular portion 27 extends entirely around the perimeter of the hollow body 11 and is located further towards the second end than the resilient arms 18.

A flange 28 is provided on the outer surface of the hollow body 11 and extends radially outwardly in a direction perpendicular to the central axis X-X. In the exemplary embodiment shown, the flange 28 is located at the proximal-most region of the first proximal end of the hollow body 11.

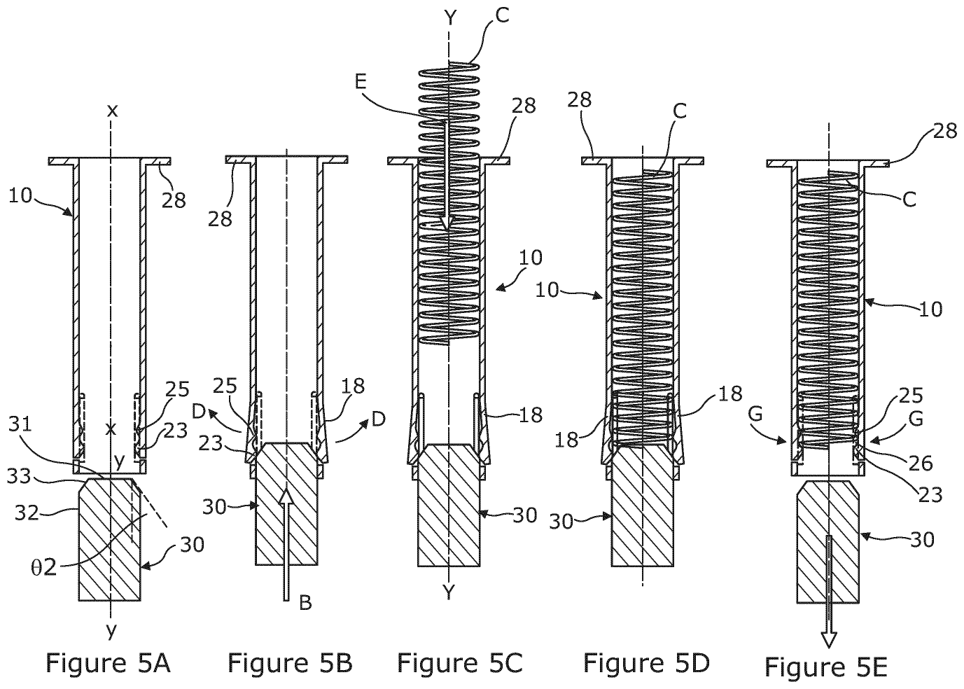

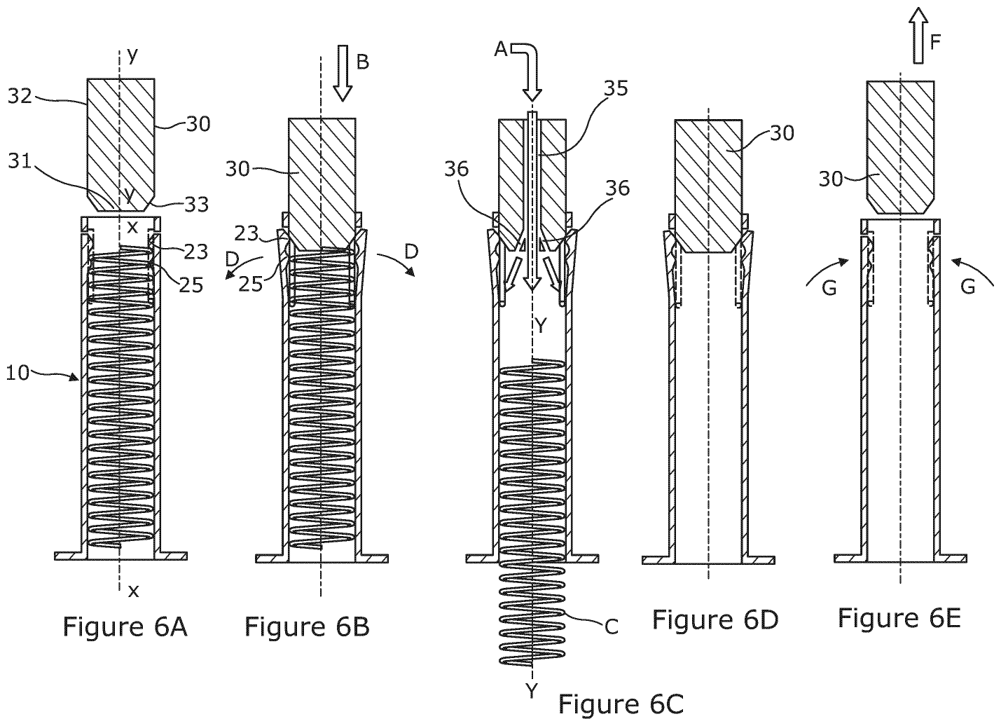

In use during a manufacturing and assembly process, the spring carrier 10 may be used to perform at least one of receiving, retaining, conveying, and discharging of a coil spring C. Such a manufacturing process may include, for example, a method of manufacturing a medicament delivery device in which a coil spring C may be required as a biasing member to actuate a medicament delivery mechanism or to actuate a needle safety mechanism after a medicament has been delivered. Use of the spring carrier 10 will now be described with reference to FIGS. 5A-5E and FIGS. 6A-6E.

The spring carrier 10 is intended to be used in conjunction with an actuator 30 operable to move the resilient arms 18. The actuator 30 and spring carrier 10 may comprise two components of an apparatus of the present invention. Such apparatus may comprise a spring carrier apparatus and may comprise part of an assembly system or apparatus for a medical device, and may comprise part of an assembly and/or manufacturing apparatus/system for a medicament injection device. However, the invention is not intended to be limited to the medical device field and is applicable to any technical field in which one or more springs may be required to be handled and conveyed.

The actuator 30 comprises a rod comprising a central axis Y-Y. The actuator 30 is configured to slide within the inner cavity 13 of the hollow body 11. In the exemplary embodiment shown, in which the hollow body 11 is cylindrical in cross-section, the actuator 30 is a cylindrical rod having an outer diameter that is slightly smaller than the internal diameter of the inner cavity 13. The actuator 30 includes a distal end 31 and a curved outer side surface 32. The actuator 30 includes a chamfered surface 33 between the distal end 31 and the side surface 32. The chamfered surface 33 extends at an acute angle θ2 with respect to the central axis Y-Y of the actuator, as shown in FIG. 5A. For instance, the chamfered surface 33 may be configured to extend at substantially the same acute angle θ2 with respect to the central axis Y-Y of the actuator as the angle θ1 of which the ramped contact surface 24 of the head 23 extends relative to the central axis X-X of the hollow body 11. This may allow improved engagement between the actuator 30 and head 23 during use, and reduce wear on the respective contacting surfaces during repeated use. The angle θ2 and/or the angle θ1 may be between 10 and 50 degrees, and may be between 15 and 45 degrees, and may be between 20 and 40 degrees, and may be between 25 and 35 degrees, and may be around 30 degrees.

FIGS. 5A-5E show method steps of insertion of a coil spring C into the spring carrier 10. In the first step shown in FIG. 5A, the actuator 30 is presented towards the second end 15 of the hollow body 11. The central axis Y-Y of the actuator 30 is aligned and coaxial with the central axis X-X of the hollow body 11.

In the next step shown in FIG. 5B, the actuator 30 is moved in an axial direction towards the spring carrier 10, as shown by arrow B in FIG. 5B, and the distal end 31 of the actuator 30 is inserted into the second opening 17 in the second end 15 of the hollow body 11. The actuator 30 first passes through the annular portion 27 of the hollow body 11. This can help serve to align the actuator 30 within the hollow body 11 such that the axes X-X, Y-Y of the hollow body 11 and actuator 30 remain coaxial. The continuous annular portion 27 of the hollow body 11 may also serve to provide structural strength to the spring carrier 10 around the resilient arms 18, and help the spring carrier 10 retain its shape and avoid damage through repeated operations in manufacturing and assembly processes in which it is used.

The actuator 30 then engages the resilient arms 18. Specifically, the chamfered surface 33 of the actuator 30 abuts the ramped contact surface 24 of the head 23 of each of the resilient arms 18. The actuator 30 continues to be moved axially towards the spring carrier 10 until it reaches a loading position as shown in FIG. 5B. In this position, the actuator 30 has caused the resilient arms 18 to be elastically deflected radially outwardly, as shown by arrows D in FIG. 5B. The resilient arms 18 flex about their respective fixed ends 21. The retaining formations of the projecting element 25 and notch 26 of each resilient arm 18 are therefore also caused to move outwardly as the resilient arms 18 move, and outwardly of an axial projection of the internal surface of the side walls 12 of the hollow body 11 defining the inner cavity 13. In the present disclosure, it will be appreciated that the terms “inwardly” and “outwardly” are used as being generally relative to the body of the spring carrier 10, for example relative to the central axis X-X or with respect to the hollow body 11 of the spring carrier 10. Therefore, movement of the resilient arms 18 outwardly is intended as being in a direction radially outwardly from the central axis X-X.

In the next step shown in FIG. 5C, the coil spring C is inserted into the inner cavity 13 through the first opening 16 at the first end 14 of the hollow body 11, as shown by arrow E. The coil spring C is sized with an outer diameter slightly smaller than the inner diameter of the inner cavity 13. As the resilient arms 18 are in a radially outward deflected position, the retaining formations on each resilient arm 18 are clear of the coil spring C as it is inserted into the inner cavity 13 and allowing the coil spring C to fall into the inner cavity 13 until it abuts the actuator 30 as shown in FIG. 5D.

In step 5E, the actuator 30 is then moved in an axial direction away from the spring carrier 10, as shown by arrow F. This moves the actuator 30 out of engagement with the resilient arms 18 and so the resilient arms 18 then move back to their first, rest position due to the elastic recovery of the material of the resilient arms 18, as shown by arrows G. As the resilient arms 18 reach the first, relaxed position, the retaining formations engage the coil spring C. That is, an end coil of the coil spring C is received within the notch 26 of each resilient arm 18, and the projecting element 25 is received between end coils of the coil spring C. The coil spring C is thereby securely retained within the spring carrier 10 and can be conveyed within the spring carrier 10 to a location and manufacturing/assembly apparatus where the coil spring C is to be utilised.

The extraction process of the coil spring C from the spring carrier 10 will now be described with reference to FIGS. 6A-6E. Before the extraction process starts, and at a preceding step in the assembly or manufacturing process requiring the coil spring C, the spring carrier 10 is inverted from the orientation shown in the insertion method steps, so that the spring carrier 10 is oriented with the first end 14 lowermost and the second end 15 uppermost. The spring carrier 10 is also positioned directly above a location where the coil spring C is to be deposited for the respective assembly/manufacturing process. For instance, the spring carrier 10 may be aligned vertically for the extraction process. This may help the coil spring C be extracted consistently and squarely from the spring carrier 10, that is, in a direction aligned with the central axis X-X of the hollow body 11.

The extraction process is generally the reverse of the insertion process described above. In the first step shown in FIG. 6A, the spring carrier 10 is oriented substantially vertically with the first end 14 lowermost, and the actuator 30 is presented vertically from above, and moved downwards towards the second end 15 of the hollow body 11. The central axis Y-Y of the actuator 30 is aligned and coaxial with the central axis X-X of the hollow body 11.

In the next step shown in FIG. 6B, the actuator 30 is moved in an axial direction towards the spring carrier 10, as shown by arrow B, and the distal end 31 of the actuator 30 is inserted into the second opening 17 in the second end 15 of the hollow body 11. The actuator 30 first passes through the annular portion 27 of the hollow body 11, which again can help to align the actuator 30 within the hollow body 11 such that the axes X-X, Y-Y of the hollow body 11 and actuator 30 remain coaxial.

The actuator 30 then engages the resilient arms 18. Specifically, the chamfered surface 33 of the actuator 30 abuts the ramped contact surface 24 of the head 23 of each of the resilient arms 18. The actuator 30 continues to be moved axially towards the spring carrier 10 until it reaches a release position as shown in FIG. 6B. In this position, the actuator 30 has caused the resilient arms 18 to be elastically deflected radially outwardly, as shown by arrows D. The resilient arms 18 flex about their respective fixed ends 21. The retaining formations of the projecting element 25 and notch 26 of each resilient arm 18 are therefore also caused to move outwardly and out of engagement with the coil spring C.

In the next step shown in FIG. 6C, since the resilient arms 18 are in a radially outward deflected position, the retaining formations on each resilient arm 18 are clear of the coil spring C and so the coil spring C is free to fall under its own weight out of the inner cavity 13 through the first opening 16 at the first end 14 of the hollow body 11. The coil spring C falls into the required location outside the spring carrier 10 so that the coil spring C is fully extracted from the spring carrier, as shown in FIG. 6D.

In step 6E, the actuator 30 is then moved in an axial direction away from the spring carrier 10, as shown by arrow F. This moves the actuator 30 out of engagement with the resilient arms 18 and so the resilient arms 18 then move back to their first, rest position due to the elastic recovery of the material of the resilient arms 18, as shown by arrows G. The spring carrier 10 may then be collected and returned to be reused in a subsequent spring insertion and extraction process.

In both the insertion process and extraction process, the spring carrier 10 may be accurately aligned with the location from and to which the coil spring C is to be inserted/extracted to allow the coil spring C to be effectively conveyed as desired and not snag on ends of the spring carrier 10 or apparatus into which the coil spring C is to be discharged. In this way, manufacturing errors and/or production stoppages to correct the error can be reduced or avoided. The flange 28 may help avoid such misalignment problems by provided a locating guide for the spring carrier in use. For example, the flange 28 may be located within a correspondingly-shaped recess in assembly/manufacturing apparatus before the coil spring C is inserted or extracted which can allow the central axis X-X of the hollow body 11 to be coaxial with the central axis of the coil spring C.

A spring carrier 10 of another embodiment of the invention is shown in FIGS. 7A-10B, and features in common with the embodiment of spring carrier 10 described previously retain the same reference numerals and will not be described again in detail.

The spring carrier 10 shown in FIGS. 7A-10B has a different configuration of head 23 at the free ends 22 of the resilient arms 18. The heads 23 include ramped contact surfaces 24 as with the previous embodiment. However, the heads 23 also include a more squared abutment step 34. The abutment step 34 extends from the ramped contact surface 24 to the inner face of the respective resilient arm 18. The abutment step 34 in the exemplary embodiment of FIGS. 7A-10B faces the first end 14 of the hollow body 11. In the relaxed position of the resilient arms 18 (see FIGS. 8A and 8B and 10A and 10B), in which the resilient arms 18 lie substantially parallel with the central axis X-X of the hollow body 11, and substantially flush with the side wall 12 of the hollow body 11, the abutment surfaces 34 extend in a plane substantially perpendicular to the central axis X-X of the hollow body 11. Whereas the equivalent abutment step surface of the first-described embodiment is angled, for example at an acute angle, with respect to the central axis X-X of the hollow body 11.

The function of the abutment step 34 will be described below with reference to FIGS. 7A-8B. FIG. 7A is equivalent to the operation step of FIG. 5D of the previously-described embodiment—that is the coil spring C has been inserted into the inner cavity 13 of the hollow body 11 and the actuator 30 is still in the loading position in which the resilient arms 18 are deflected radially outwardly. FIG. 7B shows an enlarged view of a portion of FIG. 7A, which more clearly illustrates the head 23 and abutment step 34 of a resilient arm 18, and the actuator 30 having its chamfered surface 33. During the insertion step of the coil spring C into the inner cavity 13, the coil spring C is free to fall down into the inner cavity 13. The actuator 30 deflects the resilient arms 18 outwardly such that the projecting elements 25 are moved clear of the coil spring C. That is, the resilient arms 18 may be deflected sufficiently radially outwardly by the actuator 30 that the inner-most portion of the projecting elements 25 is disposed radially outwardly of the plane of the inner surface of the side wall 12 of the hollow body 11. As such, the coil spring C is free to move past the projecting elements 25. This is illustrated particularly in FIG. 7B by line L2 which extends parallel to the central axis X-X of the hollow body 11, and intersects the radially inner-most portion of the projecting element 25. The line L2 can be seen to be disposed radially outwardly of the radial outermost portion of the coil spring C.

In the deflected position of the resilient arms 18, the head 23 and abutment step 34 extends radially inwardly further than the radially inner-most portion of the respective projecting element 25. This can be seen particularly in FIG. 7B by line L1, which extends parallel to the central axis X-X of the hollow body 11, and intersects the radially inner-most portion of the head 23/abutment step 34. The line L1 can be seen to be disposed radially inwardly of the line L2.

The line L1 can also be seen to be disposed radially inwardly of the radial outermost portion of the coil spring C. As such, even in the deflected position of the resilient arms 18, the coil spring C cannot pass the head 23 of the resilient arms and instead is blocked by contact with the abutment surface 34 of the respective resilient arms 18.

In the embodiment shown in FIGS. 7A and 7B, in the deflected position of the resilient arms 18, the distal end surface 31 of the actuator 30 is substantially level with the abutment surfaces 34, with respect to a direction perpendicular to the central axis X-X of the hollow body 11. Insertion of the coil spring C may therefore involve the coil spring C abutting both the abutment surfaces 34 and the distal end surface 31 of the actuator 30. Alternatively, in the deflected position of the resilient arms 18, the distal end surface 31 of the actuator 30 may not be level with the abutment surfaces 34, such that insertion of the coil spring C may involve the coil spring C only abutting the abutment surfaces 34 and not contacting the distal end surface 31 of the actuator 30.

Once the actuator 30 is moved away from the spring carrier 10, the resilient arms 18 move back to the first, relaxed position due to the elastic relaxing of the material of the resilient arms 18, and the coil spring C remains resting on the abutment surfaces 34, as shown in FIGS. 8A and 8B. FIG. 8A is equivalent to the operation step of FIG. 5E of the previously-described embodiment—that is the coil spring C has been inserted into the inner cavity 13 of the hollow body 11 and the actuator 30 has moved out of engagement with the resilient arms 18. FIG. 8B shows an enlarged view of a portion of FIG. 8A. Also in this position, the retaining formations of the resilient arms 18 engage the coil spring C to retain the coil spring C within the inner cavity 13 during subsequent movement of the spring carrier 10.

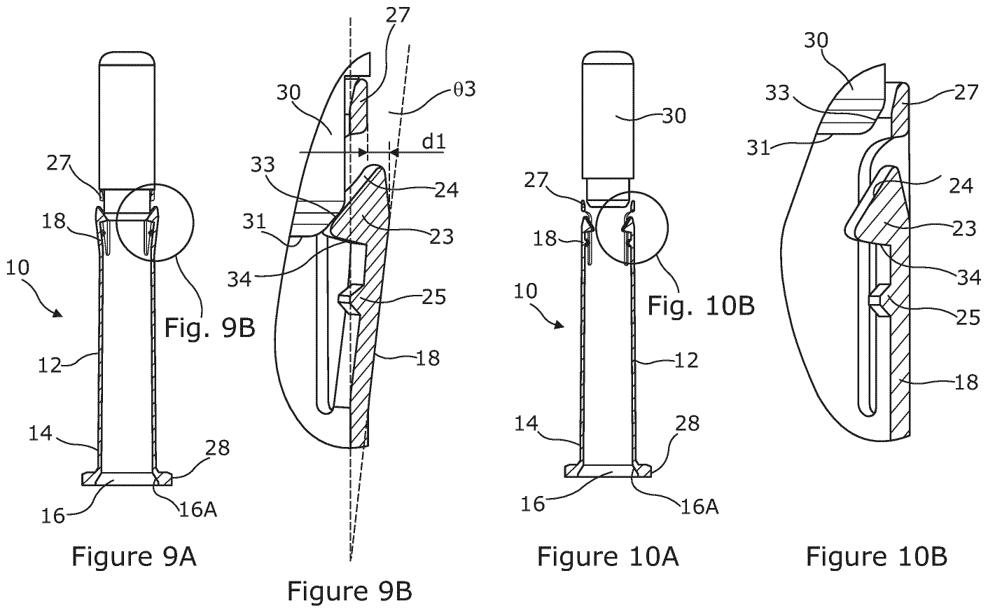

The coil spring C extraction process is shown in FIGS. 9A to 10B. FIG. 9A is equivalent to the operation step of FIG. 6D of the previously-described embodiment—that is the coil spring C has been extracted from the inner cavity 13 of the hollow body 11 and the actuator 30 is still in the extraction position in which the resilient arms 18 are deflected radially outwardly. FIG. 9B shows an enlarged view of a portion of FIG. 9A, which more clearly illustrates the head 23 and abutment step 34 of a resilient arm 18, and the actuator 30 having its chamfered surface 33.

Once the actuator 30 is moved away from the spring carrier 10, the resilient arms 18 move back to the first, relaxed position due to the elastic relaxing of the material of the resilient arms 18, as shown in FIG. 10A. FIG. 10A is equivalent to the operation step of FIG. 6E of the previously-described embodiment—that is the coil spring C has been extracted from the inner cavity 13 of the hollow body 11 and the actuator 30 has moved out of engagement with the resilient arms 18, and the spring carrier 10 may then be collected and returned to be reused in a subsequent spring insertion and extraction process. FIG. 10B shows an enlarged view of a portion of FIG. 10A.

As shown in FIGS. 7B and 9B, when the actuator 30 is inserted into the spring carrier 10 in the loading and/or extraction position, the resilient arms 18 are deflected radially outwardly by an angle θ3 from the relaxed position in which the resilient arms 18 lie substantially parallel with the central axis X-X of the hollow body 11 and substantially flush with the side wall 12 of the hollow body 11. Angle θ3 can vary within the scope of the invention, and/or within any embodiment of the invention described herein, and may vary depending on various dimensions, such as length of resilient arms 18, distance head 23 extends inwardly from resilient arm 18, diameter of hollow body 11, etc. However, the angle θ3 may be between around 4 to 12 degrees, and may be between 6 to 10 degrees, and may be around 8 degrees. This may allow sufficient deflection of the resilient arms 18 to achieve the above-described function, without excessively fatiguing the material of the resilient arms 18 and/or hollow body 11. That is, the resilient arms 18 are able to be repeatedly elastically deflected and return to the same relaxed position, without reaching the ductile limits of the material of the spring carrier 10 which would affect the ability for the resilient arms 18 to restore themselves to the intended, first, relaxed position. Contributing factors for the desired resilient performance of the resilient arms 18 include dimensions of the length, thickness and width of the resilient arms 18, and also the elastic modulus, elasticity limit and tenacity (deformation resistance at speed) of the resilient arms. Within any embodiment of the invention described herein, a range of elastic modulus may be between 1800-2500 MPa, and an elasticity limit may be 40-80 MPa. Furthermore, a tenacity of the resilient arms 18 may be 150-300 J/m². In use, to allow repeated elastic deformation and limit the aging effect on the material of the resilient arms 18, the arms may be deflected only to 40-80% of the maximum elastic limit.

FIG. 9B also shows a lateral outward deflection distance d1. This is the distance the resilient arms 18 deflect outward in the deflected position from the relaxed position, in which the outer surface of the resilient arms 18 lie flush with an outer surface of the side wall 12 of the hollow body 11. Such deflection distance d1 may vary within the scope of the invention, and/or within any embodiment of the invention described herein, but may be between 1 mm-4 mm, and may be between 1 mm-3 mm, and may be between 1-2 mm, and may be around 1.5 mm.

The embodiment of spring carrier 10 shown in FIGS. 7A to 10B includes a tapered region 16A at the first opening 16 at the first end 14. This may help guide the coil spring C into the first opening 16 during the insertion step described above. Such feature may optionally be applicable to and provided with any embodiment of the invention described herein.

Exemplary embodiments of spring carrier apparatuses comprising a spring carrier 10 and actuator 30 are described above. A variant of the above-described spring carrier apparatuses may include means for facilitating extraction of the coil spring C from the spring carrier 10. Such a variant will be described with reference to FIGS. 6A-6E. In the above-described extraction process with reference to FIGS. 6A-6E, at the step shown in step 6C, once the resilient arms 18 are in a radially outward deflected position and the retaining formations on each resilient arm 18 are clear of the coil spring C, the coil spring C falls under its own weight out of the inner cavity 13 through the first opening 16 at the first end 14 of the hollow body 11. In a variant of the above-described apparatus, the apparatus may include an air flow source or air jet A to generate a flow of air through the inner cavity 13 to blow the coil spring C out of the spring carrier 10. The actuator 30 may include an air passage 35 extending therethrough, with at least one open end at the distal end 31 of the actuator 30, and another end of the air passage 35 connected or connectable to a source of pressurised air A. In use, the air source A may be connected, or turned on, as the actuator 30 moves the resilient arms 18 into the deflected position, to send a flow of air (shown by arrows A in FIG. 6C) through the air passage 35 and out of the distal end 31 of the actuator 30. The air flow may then catch the coil spring C and force the coil spring C out of the spring carrier 10.

The actuator 30 may include a plurality of air passages 35 extending therethrough, and/or the actuator 30 may include a plurality of air passage outlets 36 at the distal end 31 of the actuator 30. The air passage(s) 35 and/or air flow outlets 36 may be aligned substantially parallel with the central axis Y-Y of the actuator 30. In addition, or alternatively, one or more air flow outlets 36 and/or air flow passages 35 may be oriented at an angle with respect to the central axis Y-Y of the actuator 30. In the latter case, the angled air flow outlets 36/passages 35 may encourage the air flow to impinge on the coils of the coil spring C to encourage expulsion of the coil spring C from the spring carrier 10. In an embodiment in which a central axial air flow passage 35/outlet 36 is provided, turbulence of air flow through the coil spring may still cause sufficient impinging of the air flow on the coils of the coil spring C to encourage expulsion of the coil spring C from the spring carrier 10.

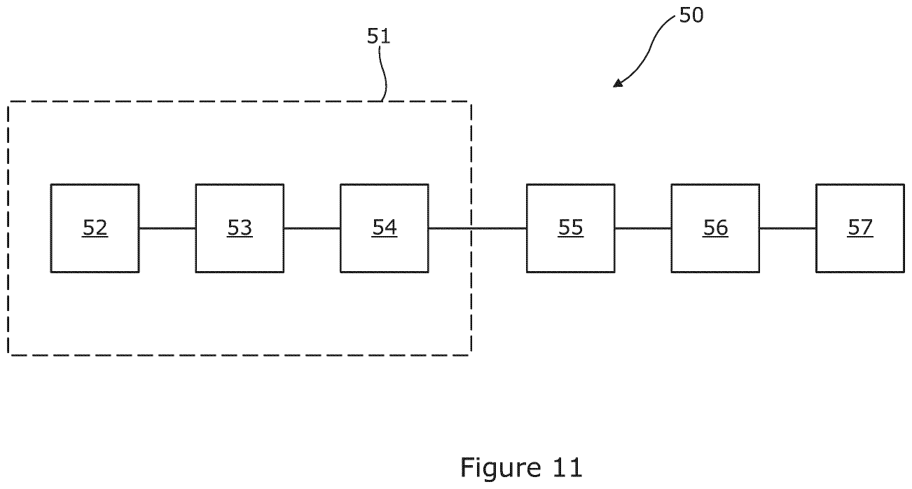

The spring carrier 10, and apparatus comprising the spring carrier 10 and actuator 30, may be part of a larger assembly system or apparatus for manufacturing devices which include one or more coil springs C. Such system may comprise a plurality of assembly machines or stations. Such assembly machines/stations may be configured as an inline process and as two or more separate processes. An exemplary assembly system 50 is shown schematically in FIG. 11. The assembly system 50 includes a coil spring making system, generally designated 51. The coil spring making system 51 may include a coiling station 52 which creates the coil spring C, a heating station 53 where the coiled spring is heated to temper the material of the spring. The heated coiled spring is then fed to a cooling station 54 to cool the coiled spring. Thereafter, a conveyor 55 transfers the cooled coiled spring C to an insertion station 56 which includes the apparatus comprising the actuator 30 and with which the spring carrier 10 may be provided. At the insertion station 56, the actuator 30 and spring carrier 10 are operated as described above to insert the coil spring C into the spring carrier 10. The spring carrier 10 with coil spring C retained therein is conveyed to an extraction station 57. At the extraction station 57, the actuator 30 and spring carrier 10 are operated as described above to extract the coil spring C from the spring carrier 10 for use in subsequent device assembly steps in which the coil spring C is utilised.

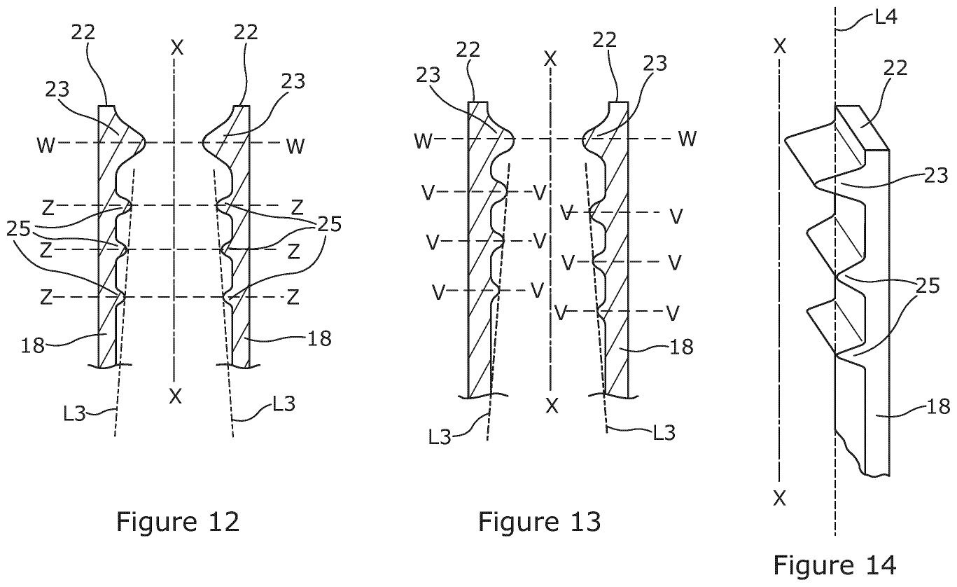

The configuration and arrangement of projecting elements 25 on the resilient arms 18 may vary within the scope of the invention, and such variants intended within the scope of the invention, and/or within the scope of all embodiments described herein, are illustrated as non-exhaustive examples in FIGS. 12 to 14.

FIG. 12 shows a schematic cross-sectional view of a configuration of one embodiment, and shows opposing resilient arms 18 in a relaxed state, each comprising a head 23 and a plurality of projecting elements 25 spaced in an axial direction of each resilient arm. The projecting elements 25 of one resilient arm 18 are aligned in an axial direction of the spring carrier 10 with the corresponding projecting elements 25 of the opposing resilient arm 18. This is shown by reference lines Z-Z, which extend through each projecting element 25 of one resilient arm 18 in a direction perpendicular to the axis X-X of the spring carrier 10, extending through the corresponding projecting element 25 on the opposite resilient arm 18. This may help securely retain a coil spring C with minimal axial movement by encouraging opposing projecting elements 25 to abut against and clamp a region of the coil spring C when retained within the spring carrier 10. This arrangement may optionally be applicable in embodiments of spring carrier 10 of the invention which comprise two resilient arms 18, or more than two resilient arms 18.

In the embodiment of FIG. 12, the head 23 of one resilient arm 18 is also aligned in an axial direction of the spring carrier 10 with the corresponding head 23 of the opposing resilient arm 18. This is shown by reference line W-W, which extend through the head 23 of one resilient arm 18 in a direction perpendicular to the axis X-X of the spring carrier 10, extending through the corresponding head 23 on the opposite resilient arm 18. This may help ensure accurate and simultaneous deflection of each resilient arm 18 upon actuation by the actuator 30 as described above.

In the embodiment of FIG. 12, the plurality of projecting elements 25 on each resilient arm 18 increase in size incrementally towards the free end 22 of each resilient arm 18. That is, the distance each projecting element 25 projects inwardly towards the central axis X-X of the spring carrier 10 is greater the closer to the free end 22 of the resilient arm 18 each is located. This is shown by lines L3 aligned with the inner-most portion of each projecting element 25 being angled inwardly towards the central axis X-X in a direction towards the free ends 22 of the resilient arms 18. This may help securely retain a coil spring C within the spring carrier 10, as larger, more inwardly-extending projecting elements 25 may be provided towards the free ends 22 of the resilient arms, yet as the free ends 22 are deflected laterally outwardly by a greater distance than a region of each resilient arm 18 spaced from the free end 22 when actuated by the actuator 30 as described above, the larger projecting elements 25 are still moved sufficiently outwardly to permit insertion of the coil spring C.

FIG. 13 shows a schematic cross-sectional view of a configuration of another embodiment, similar to that of FIG. 12, and in which like features retain the same reference numerals. The opposing resilient arms 18, each comprising a head 23 and a plurality of projecting elements 25 spaced in an axial direction of each resilient arm. A difference in the embodiment of FIG. 13 is that the projecting elements 25 of one resilient arm 18 are not aligned in an axial direction of the spring carrier 10 with the corresponding projecting elements 25 of the opposing resilient arm 18 but instead are off-set in an axial direction of the spring carrier 10 with respect to the corresponding projecting elements 25 of the opposing resilient arm 18. This is shown by reference lines V-V, which extend through each projecting element 25 of one resilient arm 18 in a direction perpendicular to the axis X-X of the spring carrier 10, not being aligned with those lines V-V through the corresponding projecting element 25 on the opposite resilient arm 18. This may help securely retain a coil spring C with minimal axial movement, and/or with axial alignment, by the staggered opposing projecting elements 25 following the helical coil of the coil spring C when retained within the spring carrier 10. This arrangement may optionally be applicable in embodiments of spring carrier 10 of the invention which comprise two resilient arms 18, or more than two resilient arms 18.

In the embodiment of FIG. 13, the head 23 of one resilient arm 18 is aligned in an axial direction of the spring carrier 10 with the corresponding head 23 of the opposing resilient arm 18. As in FIG. 12, this is shown by reference line W-W, which extend through the head 23 of one resilient arm 18 in a direction perpendicular to the axis X-X of the spring carrier 10, extending through the corresponding head 23 on the opposite resilient arm 18, with the same advantages described above.

In the embodiment of FIG. 13, the plurality of projecting elements 25 on each resilient arm 18 increase in size incrementally towards the free end 22 of each resilient arm 18, as described above with reference to FIG. 12. This is shown in FIG. 13 by lines L3 aligned with the inner-most portion of each projecting element 25 being angled inwardly towards the central axis X-X in a direction towards the free ends 22 of the resilient arms 18. This may provide the same advantages described above with reference to FIG. 12.

FIG. 14 is a schematic view of a configuration of a resilient arm 18 of another embodiment, and is similar to those of FIGS. 12 and 13. The embodiment of FIG. 14 differs in that the plurality of projecting elements 25 on each resilient arm 18 are of the same size. That is, the distance each projecting element 25 projects inwardly towards the central axis X-X of the spring carrier 10 is the same. This is shown by line L4 aligned with the inner-most portion of each projecting element 25 being parallel with the central axis X-X of the spring carrier 10. This may help securely retain a coil spring C within the spring carrier 10, as in the relaxed position of the resilient arms 18, each projecting element 25 projects equally to engage and secure a coil spring C within the spring carrier 10.

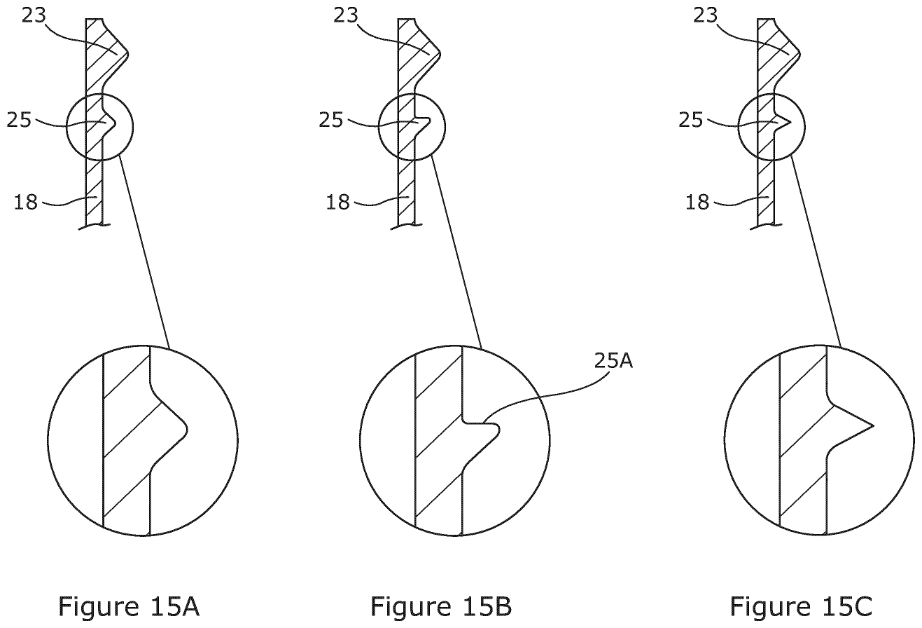

FIGS. 15A to 15C are schematic enlarged views of resilient arms 18 of spring carriers 10 of embodiments of the invention, showing different configurations of projecting elements 25 intended to fall within the scope of the invention and optionally applicable to all embodiments described herein. FIG. 15A shows a projecting element 25 comprising a generally rounded shape, with curved edges where the projecting element 25 extends from the resilient arm 18, and at the axially inner-most region of the projecting element 25. Such configuration may facilitate insertion of a coil spring C into the spring carrier 10, for example by allowing a coil spring C to ride over the projecting elements 25 if it should contact them during insertion when the resilient arms 18 are deflected outwardly, to allow the coil spring C to move to the fully inserted position.

The projecting element 25 of FIG. 15B is arranged with one surface 25A extending substantially perpendicular to the resilient arm 18 and to the axis X-X of the spring carrier 10. It is intended that such a surface 25A may be provided facing either the first end 14, or second end 16 of the spring carrier 10 within the scope of the invention. Yet further, within the scope of the invention, the projecting elements 25 may be configured with two such surfaces 25A extending substantially perpendicular to the resilient arm 18 and to the axis X-X of the spring carrier 10, one surface 25A facing the first end 14 and a second such surface 25A facing the second end 16 of the spring carrier 10. Such configuration may facilitate retention of a coil spring C within the spring carrier 10 in a desired axial position, as axial movement of the coil spring C would be more limited due to the perpendicular shape of the surfaces 25A.

FIG. 15C shows a projecting element 25 comprising a generally angled shape, with straight edges meeting at an angle where the projecting element 25 extends from the resilient arm 18, and at the axially inner-most region of the projecting element 25. Such configuration may facilitate engagement of a coil spring C within the spring carrier 10, for example by allowing the pointed edges of the projecting elements 25 to more easily located between coils of a coil spring C when the resilient arms 18 are released by the actuator to return to their relaxed position.

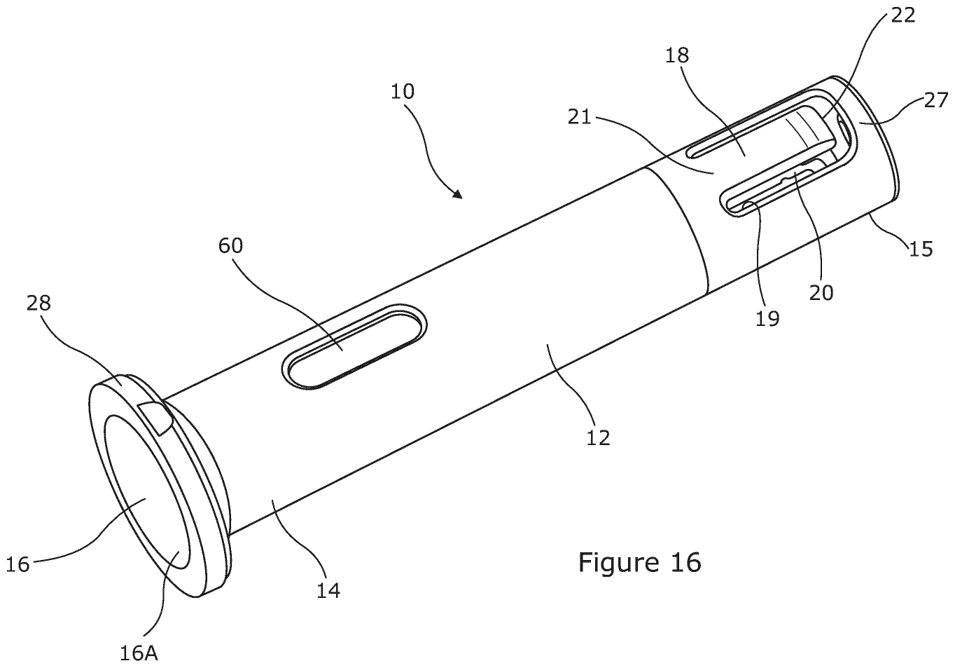

FIG. 16 shows a spring carrier 10 of another embodiment of the invention, and like features retain the same reference numerals and detailed description thereof will not be repeated. As with the embodiment of FIGS. 7A to 10B, the first opening 16 at the first end 14 includes a tapered region 16A with the advantages described above. A difference with the embodiment of FIG. 16 is that a window or cut-out region 60 is provided in and extending through the side wall 12 of the hollow body 11. This enables the interior of the hollow body 11 to be viewed from the outside of the spring carrier 10. Particularly, this enables a coil spring C to be seen when received within the spring carrier 10. This may benefit use of the spring carrier 10 in a manufacturing process. For example, in a quality control or performance monitoring process, the presence of a coil spring C within the spring carrier 10 may be checked for each device being produced. For example, an optical sensor or camera may check for the presence of a coil spring C within the spring carrier 10 and may operate using the window 60 to make such checks. For example, if it is detected that a coil spring C is absent from the spring carrier 10 due to an insertion fault elsewhere in the manufacturing process, the device being produced will likely not function correctly without the required coil spring C and so can be automatically rejected from the production line. One window 60 may be provided, or a plurality of windows may be provided, and maybe disposed in any suitable location on the side wall 12 of the spring carrier 10. The windows 60 also mean that less material is required to manufacture each spring carrier 10, which may reduce cost of manufacture and/or may also reduce the weight of the spring carrier which may be beneficial in the device manufacturing processes in which the spring carrier is to be used.

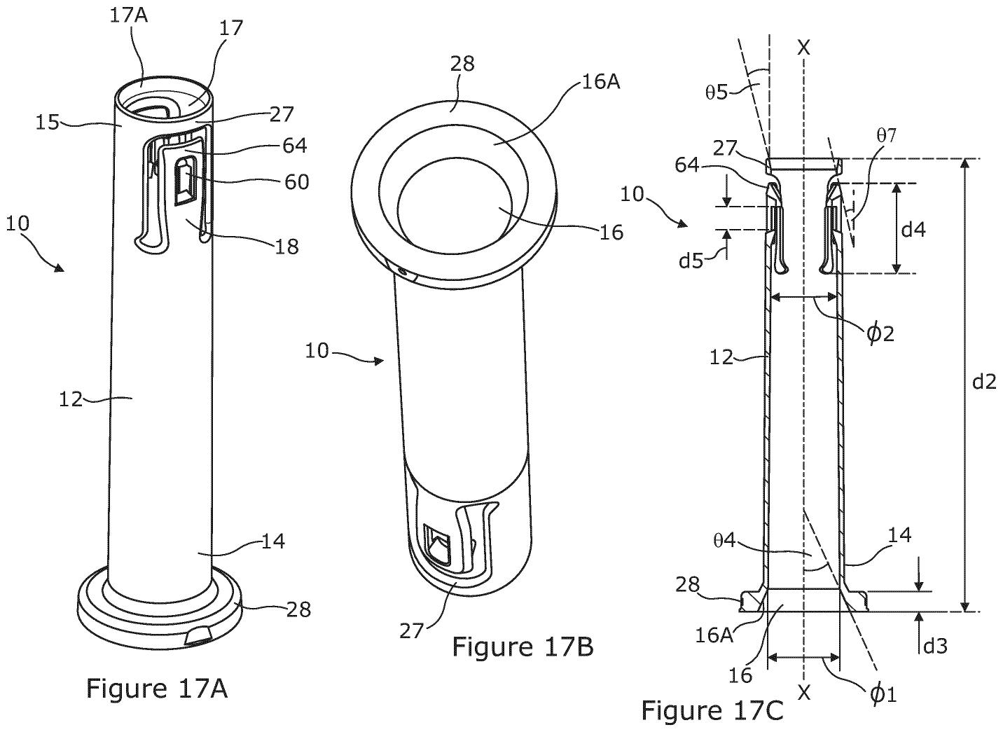

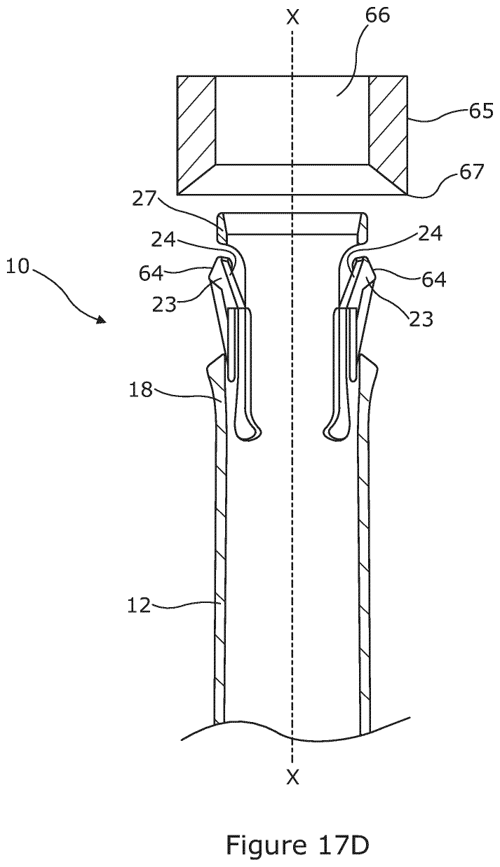

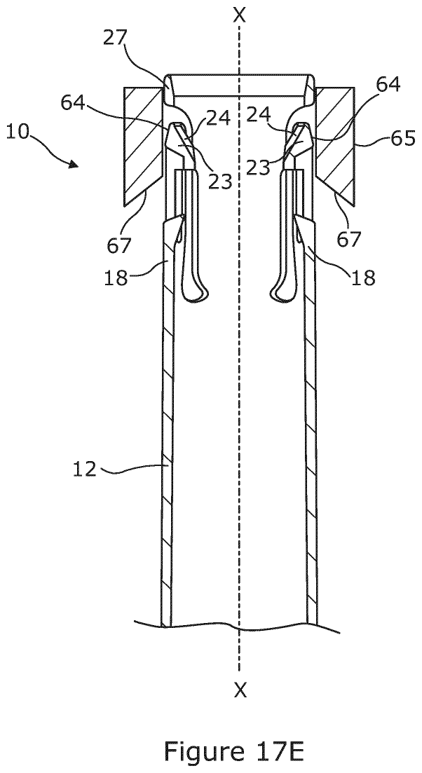

FIGS. 17A to 17E show a spring carrier 10 of another embodiment of the invention, and like features retain the same reference numerals and detailed description thereof will not be repeated. As with the embodiment of FIGS. 7A to 10B and 16, the first opening 16 at the first end 14 includes a tapered region 16A with the advantages described above. The spring carrier 10 also includes a tapered region 17A in the second opening 17 at the second end 15. This may additionally facilitate insertion and alignment of the actuator 30 in the second opening 17 during use. As can be seen from FIG. 17C, the tapered region 16A of the first opening 16 extends at an angle θ4 with respect to the axis X-X of the spring carrier 10. The angle θ4 may vary within the scope of the invention but may be between 10 to 40 degrees, and may be between 15 to 35 degrees, and may be around 24 degrees. Also as can be seen from FIG. 17C, the tapered region 17A of the second opening 17 extends at an angle θ5 with respect to the axis X-X of the spring carrier 10. The angle θ5 may vary within the scope of the invention but may be between 3 to 20 degrees, and may be between 5 to 15 degrees, and may be around 10 degrees.

It is intended within the scope of the invention, and within any of the embodiments described herein, that one or other, or both of the first opening 16 at the first and 14 and second opening 17 at the second end 15 may include such tapered region 16A, 17A, for the advantages described above, and of any of the dimensions described above.

Another difference with the embodiment of FIGS. 17A to 17C over that of FIG. 16 is that although a window 60 is provided, it is provided in the resilient arm 18 instead of the side wall 12 of the hollow body 11. The window 60 still provides the advantage described above of being able to detect the presence of a coil spring C within the spring carrier 10. However, the window 60 may also render the resilient arm 18 lighter and/or more readily deflectable than if the window 60 was not provided in the resilient arm 18. This may require less actuator force to deflect the resilient arm 18 by the required amount in use of the spring carrier 10. Such reduced forces may reduce the stress on the material of the spring carrier 10 and enable greater life cycle of the spring carrier 10 before failure or replacement is required.

Another feature of the embodiments of FIGS. 17A to 17D is that a restoring surface 64 is provided on an outer region of the resilient arms 18. The restoring surface 64 is an angled surface which is directed radially inwardly towards the central axis X-X of the hollow body 11 in a direction towards the second, distal end 15. The restoring surface 64 may help the resilient arms 18 be returned fully back to the first, unbiased position in which the resilient arms 18 lie flush with an outer surface of the hollow body 11 of the spring carrier 10, as described in more detail below with reference to FIGS. 17D and 17E.

FIG. 17D shows an enlarged view of the second, distal end 15 of the spring carrier 10 shown in FIG. 17C. However, in FIG. 17D, the resilient arms 18 are shown displaced slightly outwardly from the first unbiased position and so not flush with an outer surface of the hollow body 11 of the spring carrier 10. Repeated use of the spring carrier 10 and repeated deflection of the resilient arms 18 may cause fatigue in the material of the resilient arms 18. This may result in the resilient arms 18 not returning fully to the desired first, unbiased position when the actuator 30 is removed. This may therefore result in a coil spring C within the spring carrier 10 not being securely retained as required. To overcome this, it may be desirable to actively return the resilient arms to the correct first, unbiased position. This may be by means of an external force being applied to the resilient arms 18 acting radially inwardly. Such force may, for example, be applied by a restoring tool 65, as shown in FIGS. 17D and 17E. The restoring tool 65 may be slid over the second, distal end 15 of the spring carrier 10 to engage the restoring surfaces 64 and push the resilient arms 18 radially inwardly to the first unbiased position, as shown in FIG. 17E.

As shown in FIG. 17C, the restoring surfaces 64 may be disposed at an angle θ7 with respect to the central axis X-X when the resilient arms 18 are in the correct first unbiased position. This may help the resilient arm 18 progressively engage the restoring tool 65 as the restoring tool 65 is slid over the spring carrier 10 to return the resilient arm 18 to the correct first unbiased position. Such angle θ7 may vary within the scope of the present disclosure and may be between 2 to 10 degrees, and may be between 4 to 8 degrees, and may be around 6 degrees.

The restoring tool 65 may be provided with a central bore 66 to receive the spring carrier 10 as the restoring tool 65 is slid over the second distal end 15 of the spring carrier 10. The central bore 66 may be slightly larger in diameter than the outer diameter of the hollow body 11 of the spring carrier 10. The restoring tool may have an angled contact face 67 which is configured to engage the restoring surface 64 of the resilient arms 18. The angled contact face 67 of the restoring tool 65 may extend at the same angle θ7 relative to a central axis of the restoring tool 65, as the restoring surface 64 extends relative to the central axis X-X of the spring carrier 10. It is intended that the feature of the restoring surfaces 64 on the resilient arms 18 may optionally be applicable to and provided with any embodiment of the invention described herein. Furthermore, the reforming surface 64 may be engaged by means other than the restoring tool 65 described above, in order to restore the resilient arms 18 to the intended first unbiased position, whilst providing the benefits described above.

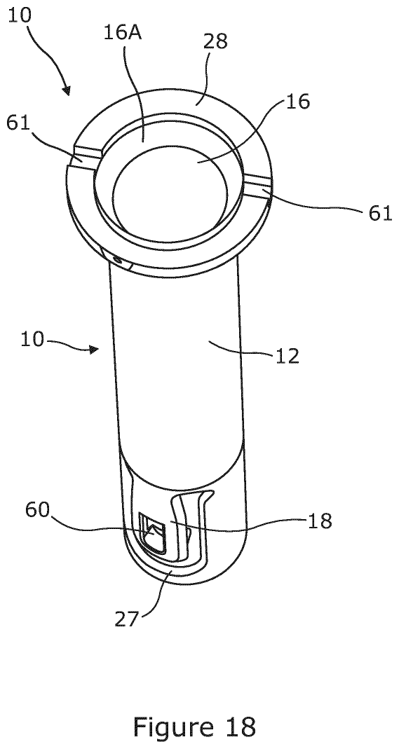

FIG. 18 shows a spring carrier 10 of another embodiment, similar to the embodiment of FIGS. 17A to 17C, and like features retain the same reference numerals and detailed description thereof will not be repeated. As with the embodiment of FIGS. 7A to 10B, the first opening 16 at the first end 14 includes a tapered region 16A with the advantages described above. A difference with the embodiment of FIG. 18 is that the flange 28 includes orientation features 61. In the exemplary embodiment shown, the orientation features 61 comprise a pair of radial slots formed into the surface of the flange 28 facing in the direction of the first end 14. Such orientation features 61 may facilitate correct rotational positioning of the spring carrier 10 about its central axis X-X which may be beneficial for function of the spring carrier in use, for example for insertion or extraction of a coil spring C, and/or insertion of the actuator 30. Furthermore, such orientation feature may be used in conjunction with the window 60 during a manufacturing process. For example, an optical sensor or camera used to detect the presence of a coil spring C within the spring carrier 10 may be located in a certain position on a manufacturing apparatus/system or assembly line, and so require correct orientation of the spring carrier 10 to align the window 60 with the optical sensor or camera. The orientation features 61 may cooperate with corresponding features (not shown) such as projections which may be received in the slots of the orientation features 61 to ensure correct positioning of the spring carrier 10 in use.

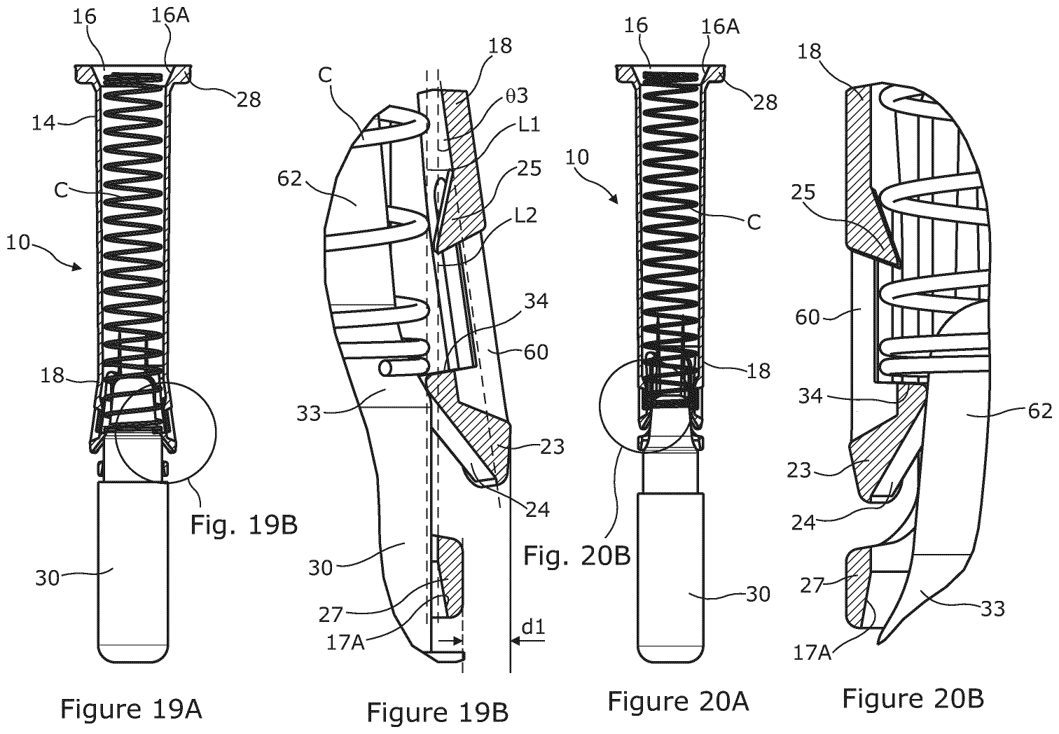

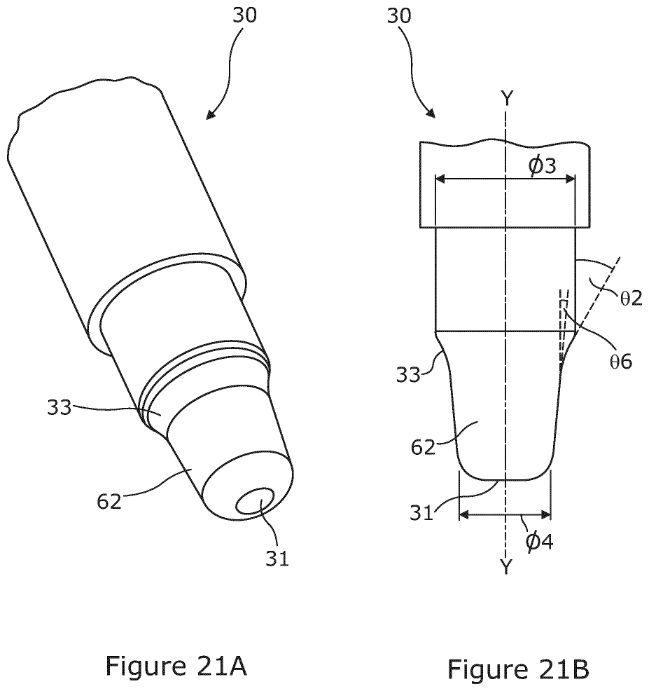

FIGS. 19A to 20B show a spring carrier 10 of another embodiment of the invention, in use with an actuator of another embodiment of the invention, shown in FIGS. 21A and 21B. Like features retain the same reference numerals and a detailed description thereof will not be repeated. FIG. 19A shows an arrangement of spring carrier 10 and actuator 30 equivalent to the operation step of FIGS. 5D and 7A of the previously-described embodiments—that is the coil spring C has been inserted into the inner cavity 13 of the hollow body 11 and the actuator 30 is still in the loading position in which the resilient arms 18 are deflected radially outwardly. FIG. 19B shows an enlarged view of a portion of FIG. 19A, which more clearly illustrates the head 23 and abutment step 34 of a resilient arm 18, and the actuator 30 having its chamfered surface 33.

As with the embodiment of 5D and 7A described previously, in the stage shown in FIGS. 19A and 19B, the actuator 30 has deflected the resilient arms 18 outwardly such that the projecting elements 25 are moved clear of the coil spring C and the coil spring C can fall into the spring carrier 10, again, illustrated in FIG. 19A by line L2 which extends parallel to the central axis X-X of the hollow body 11, and intersects the radially inner-most portion of the projecting element 25, being disposed radially outwardly of the radial outermost portion of the coil spring C. Also, the head 23 and abutment step 34 extend radially inwardly further than the radially inner-most portion of the respective projecting element 25, shown by line L1, which extends parallel to the central axis X-X of the hollow body 11 and intersects the radially inner-most portion of the head 23/abutment step 34, being disposed radially inwardly of the line L2.

In the embodiment of FIGS. 19A to 20B however, the line L1 can also be seen to be disposed approximately level with both the radial outermost portion of the coil spring C, and the end of the coil spring C when inserted within the spring carrier 10 abuts the chamfered surface 33 of the actuator 30, and is also substantially axially level with the abutment step 34 of the head 23.

A difference in the embodiment of FIGS. 19A to 20B is also that the actuator 30 comprises a narrowed section 62 at a distal end which extends beyond the chamfered surface 33. This narrowed section 62 is configured to fit within the coil spring C as can be seen in FIGS. 19A to 20B. The narrowed section 62 also permits a smooth and gradual deflection of the resilient arms 18 as the actuator is initially inserted into the second end 15 of the spring carrier 10. During insertion of the coil spring C therefore, the coil spring C slides within the inner cavity 13 and over the narrowed section 62 of the actuator 30 until the coil spring C abuts the chamfered surface 33 of the actuator 30. At this point, the coil spring C may also abut an edge of the abutment surfaces 34. Also at this point, the lowermost end of the coil spring is substantially level in an axial direction with the abutment step 34 of the head 23, as shown in FIG. 19B. Thereafter the actuator 30 is withdrawn from the spring carrier 10 as described previously and shown in FIGS. 20A and 20B, allowing the resilient arms 18 to return to their relaxed position, and the abutment surfaces 34 slide inwardly and underneath the end of the coil spring C to support the coil spring C, with the projecting elements 25 securely retaining the coil spring C within the spring carrier 10.

During an insertion process of the coil spring C, the narrowed section 62 being received within the coil spring C may help axially align the coil spring C within the spring carrier 10. This may enable more secure retaining of the coil spring C within the spring carrier 10 by the projecting elements 25, and also ensure the coil spring C is aligned for accurate extraction in a later stage of a device manufacturing process. It will be appreciated that during extraction of the coil spring C, the actuator 30 would be inserted into the second opening 17 at the second end 15, and the narrowed section 62 of the actuator 30 would be received within the coil spring C. This may further help axial alignment of the coil spring C for an accurate extraction from the spring carrier 10.

It can be seen from FIGS. 19A to 20B that the embodiment of these figures includes a number of features described previously with the technical advantages previously described with each. Such features include the tapered regions 16A, 17A of the first and second openings 16, 17 respectively. Also, the resilient arms 18 can be seen to comprise a window 60. In the deflected position of the resilient arms 18, the resilient arm extends at an angle θ3 to the axis X-X of the spring carrier 10. Such angle θ3 may be within the range of angles described above, and may be between 4 to 12 degrees, and may be between 6 to 10 degrees, and may be around 8 degrees. FIG. 19B also illustrates a maximum lateral outward deflection distance d1 of the resilient arms 18 in the deflected position from the outer surface of the side wall 12 of the hollow body 11. Such deflection distance d1 may be of the ranges of distances d1 described above.

Within the scope of the invention, and/or within any embodiment of the invention described herein, the angle θ3 of the resilient arms 18 in the deflected position relative to the axis X-X, and the maximum lateral outward deflection distance d1 of the resilient arms 18 in the deflected position from the outer surface of the side wall 12 of the hollow body 11, may differ between a configuration during insertion of a coil spring C and a configuration during extraction of a coil spring C. For example, during an insertion process, the actuator 30 may be engaged with the spring carrier 10 such that the angle θ3 and the distance d1 are less than the angle θ3 and the distance d1 during an extraction process. This may be achieved by the actuator 30 being inserted further into the second end 15 of the spring carrier 10 during an extraction process than during an insertion process. This may help ensure reliable extraction of the coil spring C from the spring carrier 10 by the further deflection of the resilient arms during extraction of the coil spring C.

In some embodiments, during insertion of a coil spring C, such angle θ3 may be between 4 to 12 degrees, and may be between 6 to 10 degrees, and may be around 8 degrees. In some embodiments, during extraction of a coil spring C, such angle θ3 may be between 7 to 15 degrees, and may be between 9 to 13 degrees, and may be around 11 degrees. In some embodiments, during insertion of a coil spring C, such deflection distance d1 of the resilient arm 18 may be between 1 mm-4 mm, and may be between 1 mm-3 mm, and may be between 1-2 mm, and may be around 1.3 mm. In some embodiments, during extraction of a coil spring C, such deflection distance d1 of the resilient arm 18 may be between 1 mm-4 mm, and may be between 1 mm-3 mm, and may be between 1-2.5 mm, and may be around 1.8 mm.

FIGS. 21A and 21B show an actuator 30, and particularly the end detail of the actuator 30. The actuator 30 comprises features previously-described, including the distal end 31, chamfered surface 33 and narrowed section 62. The angle θ2 of the chamfered section relative to the central axis Y-Y of the actuator 30 may be within the range of angles described previously, namely between 10 and 50 degrees, and may be between 15 and 45 degrees, and may be between 20 and 40 degrees, and may be between 25 and 35 degrees, and may be around 30 degrees.

The narrowed section 62 may comprise an outer side wall which extends substantially parallel to the central axis Y-Y of the actuator 30. Alternatively, the outer side wall may extend at an angle θ6 relative to the central axis Y-Y of the actuator 30 and may be configured to taper outwardly away from the distal end 31. Such angle θ6 may vary within the scope of the invention, and may be between 1 to 10 degrees, and may be between 2 to 9 degrees, and may be between 3 to 8 degrees, and may be between 4 to 7 degrees, and may be around 5 or 6 degrees. An angled outer side wall 62 of the narrowed section 62 may help insertion of the actuator both into the spring carrier 10 and/or within the coils of the coil spring C during the insertion and/or extraction process of the coil spring C.

The chamfered surface 33 may extends between the narrowed section 62 and a wider portion of the actuator. The wider portion may have a diameter Ø3 of between 7 to 13 mm, and may be between 7 to 12 mm, and may be between 9 to 11 mm, and may be around 10 mm. The narrowed section may gave a minimum outer wall diameter Ø4, before a rounding to the distal end 31, of 3.5 to 9.5 mm, and may be between 4.5 to 8.5 mm, and may be between 5.5 to 7.5 mm, and may be around 6.5 mm.

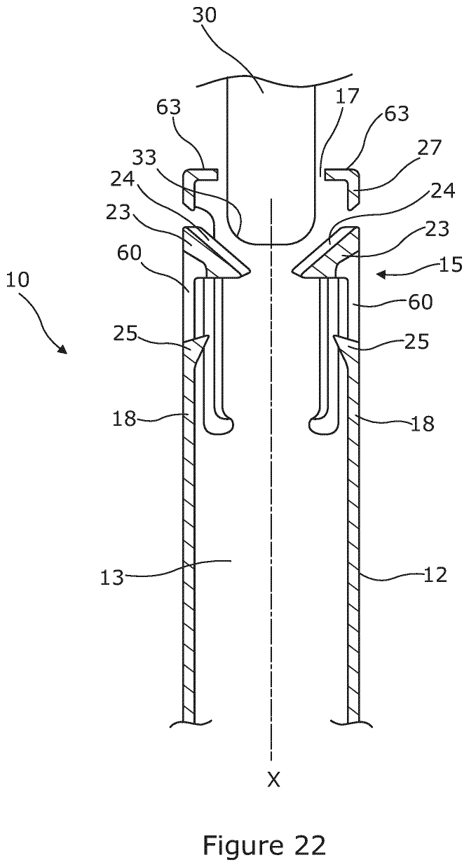

FIG. 22 shows another embodiment of a spring carrier 10 of the invention, and is similar to the spring carrier 10 shown in FIGS. 19A to 20B, although oriented the other way around in FIG. 21. Like features retain the same reference numerals and detailed description thereof will not be repeated. A difference in the embodiment of FIG. 22 is that the opening 17 at the second end 15 of the hollow body 11 is of a smaller cross-sectional dimension than the cross-sectional dimension of the inner cavity 13. That is, the distal portion of the second end 15 of the hollow body 11 includes at least one inwardly extending protrusion 63 around the circumference of the second end 15, and the second opening 17 is defined within the inner perimeter of the or each protrusion 63. Such protrusion 63 may include an inwardly protruding wall or lip around the entire circumference of the second end 15, or one or more such inwardly protruding lips around a portion of the circumference of the second end 15. The protrusion may also comprise one or more discrete protrusions spaced from each other and extending inwardly from the circumference of the second end 15. This may help provide the hollow body with increased strength and/or rigidity, particularly in the region of the second end 15. This may also serve as a blocking formation and prevent a coil spring C from being able to pass out of the second end 15 of the spring carrier 10 during an insertion step or, once a coil spring C is inserted within the spring carrier, during subsequent transport and manipulation/manufacturing steps. The spring carrier of this embodiment would still function as described previously, although it will be appreciated that an actuator 30 would need to be of reduced external diameter to be able to pass through the second opening 17. Furthermore, the heads 23 of the resilient arms 18 would need to be extended inwardly towards the central axis X-X beyond the axial projection of the inner perimeter of the lip 63 so that they may be engaged by the actuator 30 as described previously to deflect the resilient arms 18. Such extended head 23 configuration is shown in FIG. 22.

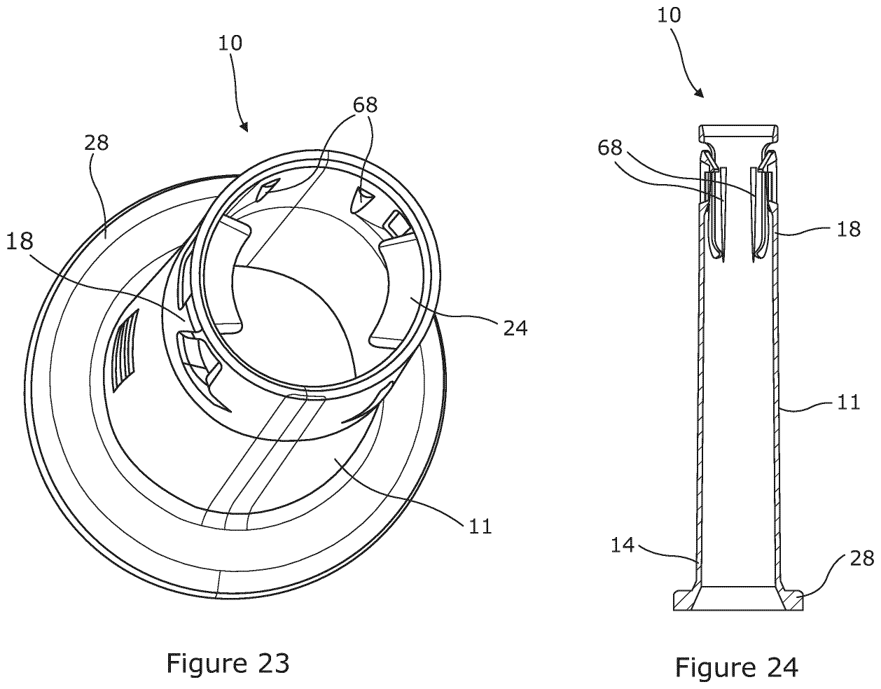

FIGS. 23 and 24 show a spring carrier 10 of another embodiment of the invention, similar to the spring carrier of FIGS. 17A-17E, and like features retain the same reference numerals and detailed description thereof will not be repeated. A difference in the spring carrier of FIGS. 23 and 24 is that the inner surface of the side wall 12 of the hollow body 11 includes a plurality of centering lugs 68 which project inwardly towards the central axis X-X of the hollow body 11. In the embodiment shown, four centering lugs 68 are provided. However, more than four or fewer than four may be provided, and the centering lugs 68 may optionally be equally spaced around the inside circumference of the side wall 12.

The centering lugs 68 are formed as ramps with a curved surface and increase in the distance they project inwardly as the centering lug 68 extends towards the second distal end 15 of the spring carrier 10. In use, the centering lugs 68 serve to contact and centre a coil spring C held within the spring carrier 10 so that the coil spring C is accurately retained centrally within the spring carrier 10. The centering lugs 68 may compensate for any tolerance between the outer diameter of the coil spring C and internal diameter of the inner cavity 13 to reduce play between the coil spring C and spring carrier 10. This may help ensure the coil spring C is accurately located during insertion of the coil spring C into the spring carrier 10, to help ensure the coil spring C can be securely engaged by the retaining formations. This may help avoid accidental or premature spring extraction during transportation of the spring carrier 10 or during a manufacturing process in which the coil spring is required to be accurately extracted and positioned into a device being manufactured. The may help prevent manufacturing errors and/or stoppages. The feature of the centering lugs 68 may optionally be applicable to and provided with any embodiment of the invention described herein.

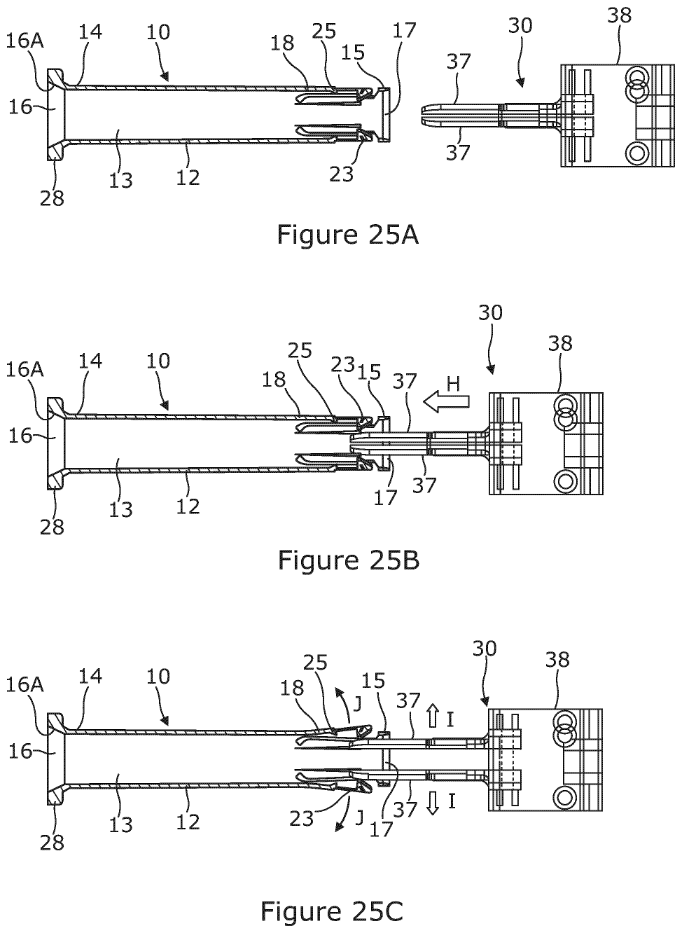

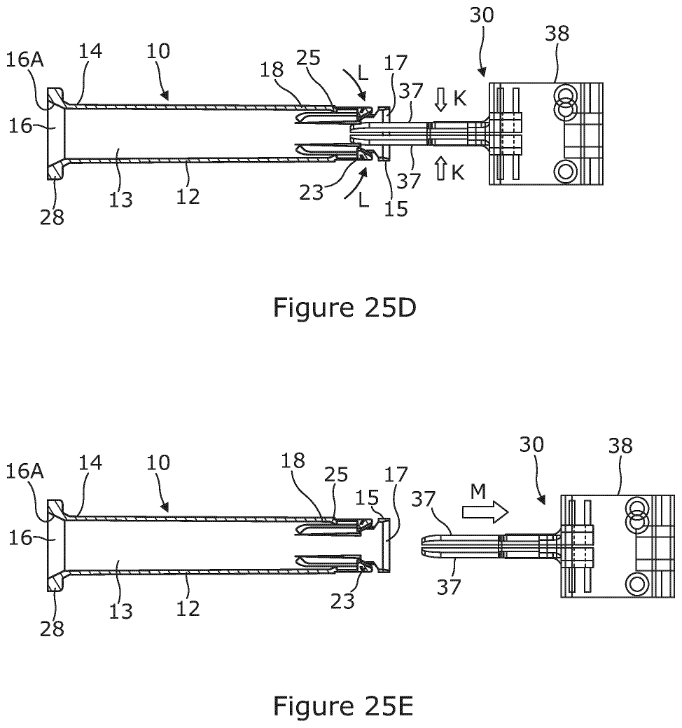

FIGS. 25A to 25E show a sequence of steps of use of a spring carrier 10 of an embodiment described previously, in use with an alternative embodiment of actuator 30. The actuator 30 comprises a pair of moveable jaws 37 which are moveable mounted to a chuck 38 so that the jaws 37 can translate towards and away from each other. The jaws 37 are moveable from a disengaged position, as shown in FIGS. 25A, 25B, 25D and 25E, to an engaged position, as shown in FIG. 25C.

In the disengaged position, the jaws 37 are disposed proximate or in contact with each other to form a rod-like shape. In the engaged position, the jaws 37 are moved away from each other in a generally radial direction of said rod shape.

In use, in step shown in FIG. 25A, the actuator 30 is initially spaced away from the second distal end 15 of the spring carrier 10, and the rod shaped actuator jaws 37 are in the disengaged position and are generally axially aligned with the central axis X-X of the spring carrier 10.

In the step shown in FIG. 25B, the actuator 30 is moved towards the spring carrier 10 in a direction shown by arrow H and the jaws 37 are inserted into the second opening 17 at the second distal end 15 of the spring carrier 10. The actuator 30 is inserted such that the remote ends of the jaws 37 are generally aligned in an axial direction of the spring carrier 10 with the heads 23 at the free ends 22 of the deflectable members 18.

At the next step shown in FIG. 25C, the jaws 37 are moved apart as shown by arrows I into the engaged position such that they engage a respective adjacent deflectable member 18. This causes the deflectable members 18 to move in the direction shown by arrows J from the first unbiased position to the second, biased position. This also moves the projecting elements 25 of each deflectable member 18 outwardly. At this stage, the coil spring C can be inserted into the spring carrier 10 through the first opening 16 at the first proximal end 14 of the spring carrier 10. The coil spring C is not shown in FIGS. 25A to 25E for ease of illustration of other features described herein.

Once the coil spring C is fully inserted such that it contacts or generally reaches close to the position of the actuator jaws 37, the jaws 37 move in the direction shown by arrows K back to the disengaged position. This allows the deflectable members 18 to return to the unbiased position shown by arrows L. In doing so, the projecting elements 25 engage the coil spring C to retain the coil spring C within the spring carrier 10.

Finally, in the step shown in FIG. 25E, the actuator is retracted away from the spring carrier 10 in the direction shown by arrow M. The coil spring C (not shown) remains securely retained within the spring carrier 10.

The above-described sequence of steps describes an exemplary coil spring insertion process using the spring carrier 10 and actuator 30 of an embodiment of the invention. It will be appreciated that a method of extraction of a coil spring C from the spring carrier 10 could comprise the reverse of the above-described method steps in use of the spring carrier 10 and actuator 30 of this embodiment of the invention.

Although in FIGS. 25A to 25E, the spring carrier 10 and actuator 30 are shown oriented generally horizontally, the apparatus and method of use is not limited to this orientation and in alternative embodiments, the spring carrier 10 and actuator may be operated in other orientations, including but not limited to vertically, such as shown in embodiments in FIGS. 5A-6E. For example, the spring carrier 10 may be oriented vertically with the first proximal end 14 uppermost during a coil spring insertion process, and may be oriented vertically with the second distal end uppermost in a coil spring extraction process.

During the coil spring insertion and extraction process, the coil spring C may be conveyed into and out of the spring carrier by various means, for example falling under its own weight, being actively driven, and by any other means described above, for example under force of a flow of air from air source.

The alternative configuration actuator 30 described above with reference to FIGS. 25A to 25E may optionally be applicable to and provided for use with any embodiment of spring carrier 10 of the invention described herein.

In the exemplary embodiment of actuator 30 shown in FIGS. 25A to 25E, the actuator 30 is described as comprising two jaws 37. However, the invention is not intended to be limited to this configuration and in alternative embodiments, the actuator may comprise one jaw 37 or may comprise more than two jaws 37. In some instances, the actuator 30 may comprise the same number of jaws 37 as number of deflectable members 18 on the spring carrier 10 which the actuator 30 is intended to operate with, such that one jaw 37 deflects each deflectable member 18.

In the exemplary embodiment of actuator 30 shown in FIGS. 25A to 25E, the actuator 30 is described moving the deflectable members by a remote end of each jaw 37 contacting the respective head 23. However, the invention is not intended to be limited to this configuration and in alternative embodiments, the actuator may be arranged or operated to engage other parts of deflectable member, other than a specific actuation feature such as the head 23 in the present example, and/or by another region of the jaw(s) 37 other than the remote end. Furthermore, it is envisaged that other means may be employed in an exemplary insertion/extraction process to effect movement of the resilient arms other than the exemplary embodiment of actuator 30 that is shown and described. Some other external operator or mechanism (not shown) may engage and move the deflectable member(s) as required. Such an embodiment may again not require engagement with a specific actuation feature, or use of the specific actuator 30 shown and described, although these are not precluded within the scope of the present disclosure.

As mentioned above, throughout the present disclosure, it will be appreciated that the terms “inwardly” and “outwardly” are used as being generally relative to the body of the spring carrier 10. For example, relative to the central axis X-X or with respect to the hollow body 11/inner cavity 13 of the spring carrier 10. As such, as used herein, the deflectable member and/or retaining formation(s) being disposed or extending “outwardly” in a second deflected position will be understood as being disposed in a direction further away from the inner cavity 13 and/or axis X-X than when in a more inwardly-disposed position in a first, unbiased position. In some embodiments, as described above, the retaining formation(s) may be disposed outwardly of an inner surface of the inner cavity 13 in a second, biased position. This may help ensure a coil spring is discharged from the inner cavity 13. However, it will be appreciated that in alternative embodiments intended within the scope of the invention, the retaining formation(s) may be disposed further outwardly in the second, biased position than in a first, unbiased position, but not disposed outwardly of an inner surface of the inner cavity 13. It may be sufficient for the retaining formation(s) to be disposed further outwardly in the second, biased position such that a gap is provided which is at least larger than a coil spring diameter to allow the coil spring to pass out of the inner cavity 13. In an exemplary embodiment in which a coil spring has a diameter which is significantly smaller than an inner diameter of the inner cavity (but large enough to be retained by the retaining formation(s) when the deflectable member(s) is in the first, unbiased position), the retaining formation(s) may not need to be deflected outwardly of an inner surface of the inner cavity 13 in the second, biased position to disengage the coil spring to allow its release.

The various embodiments of spring carrier 10 illustrated and described above are intended to be configured in a range of shapes and sizes and relative dimensions within the scope of the invention. However, exemplary dimensions are described herein with reference to the embodiment shown in FIG. 17C and dimensions marked therein.

The spring carrier 10 may comprise a total length d2 in a direction of the axis X-X of between 50 mm to 90 mm, and may be between 60 mm to 80 mm, and may be around 70.5 mm or around 73.5 mm.

The flange 28 may comprise a height d3 in a direction of the axis X-X of between 1 mm to 5 mm, and may be between 2 mm to 4 mm, and may be around 3 mm.

The resilient arms 18 may comprise a total length d4 in a direction of the axis X-X from the fixed end 21 to the free end 22 of between 10 mm to 20 mm, and may be between 12 mm to 18 mm, and may be between 14 to 16 mm, and may be around 16.3 mm.

The window 60 when provided in the side wall 12 may comprise a length in a direction of the axis X-X of between 5 mm to 25 mm, and may be between 10 mm to 20 mm, and may be around 15 mm. The window 60 when provided in the resilient arms 18 may comprise a length d5 in a direction of the axis X-X of between 1.5 mm to 8 mm, and may be between 2.5 mm to 7 mm, and may be between 3.5 to 6 mm, may be around 4.3 mm.

The hollow body 11 is shown and described as being configured as a cylindrical tube which is circular in cross-section. This allows to closely contain coil springs C of conventional circular form. This also may facilitate ease of insertion of the coil springs C, and alignment of the spring carrier 10 for extraction of the coil spring C, as no specific rotational orientation about the central axis X-X is required for correct positioning of the spring carrier 10 in use. However, the invention is not intended to be limited to such a configuration of spring carrier, and other dimensions and cross-sectional shapes are possible, such as oval, triangular or square, or other polygons.

The hollow body 11 is shown and described as being of a substantially constant cross-section along its length from the first end 14 to the second, opposite end 15. Referring to FIG. 17C, in such an embodiment, an internal diameter Ø1 in a region of the first end 14 of the spring carrier 10 would substantially equal an internal diameter Ø2 in a region of the second end 15 of the spring carrier 10. This may facilitate ease and cost of manufacture and of manipulation in assembly or manufacturing processes in which the spring carrier 10 is to be utilised. However, the invention is not intended to be limited to such a configuration and in alternative embodiments, the spring carrier 10 may vary in cross-sectional dimension along its length. For example, the cross-section may be circular of different diameters along the length of the spring carrier, and/or the cross-section may be shaped other than circular along part of the length of the spring carrier. For example, the internal diameter Ø1 may be larger than the internal diameter Ø2 such that the inner cavity is slightly wider in the region of the first end 14 of the spring carrier 10 through which the coil spring C is inserted and extracted. This may further help accurately guide the coil spring C into the spring carrier 10, in addition to assistance from the tapered region 16A. This may also allow the coil spring C to be more closely confined in the region of the second end 15 of the spring carrier 10, where the coil spring C is to be engaged and retained by the retaining formations of the projecting elements 25 and notches 26. However, the opposite may be the case within the scope of the invention and the internal diameter Ø1 may be smaller than the internal diameter Ø2 such that the inner cavity is slightly narrower in the region of the first end 14 of the spring carrier 10.

In an exemplary embodiment in which Ø1 and Ø2 are substantially equal, each may be between 7 mm to 14 mm, and may be between 8 mm to 13 mm, and may be between 9 mm to 12 m, and may be between 10 mm to 11 mm, and may be around 10.5 mm or around 11.5 mm.

In an exemplary embodiment in which Ø1 and Ø2 are unequal, one of Ø1 and Ø2 may be between 9 mm to 14 mm, and may be between 10 mm to 13 mm, and may be between 11 mm to 12 m, and may be around 11.5 mm. The other of Ø1 and Ø2 may be between 8 mm to 13 mm, and may be between 9 mm to 12 mm, and may be between 10 mm to 11 m, and may be around 10.5 mm.

Various materials may be selected from which the spring carrier 10 is formed, which includes plastics and metals, and may include various polymers, including Polypropylene, Polyester, Polyamide or Acrylo-Butadiene-Styrene (ABS). The spring carrier may further be formed from Polycarbonate, and may comprise recycled Polycarbonate.

The spring carrier 10 is shown and described as a single moulded component, that is, a single integral component. As such, the resilient arms 18 for example, are shown as being integrally formed with hollow body 11. This may provide advantages of ease and reduced cost of manufacture. However, it is intended within the scope of the invention that one or more elements of the spring carrier 10 may be separate components secured, bonded, welded, mechanically fastened together. For example, the resilient arms 18, or the flange 28, may not be integrally formed with the hollow body 11.

The side wall 12 of the hollow body 11 may be of a dimension to provide sufficient structural strength during use, but also minimise excess use of material and maintain light-weight for ease of handling and cost of manufacture. The wall thicknesses may be between 0.3 mm to 1.5 mm, for instance between 0.5 mm-1 mm in thickness.

Embodiments of spring carrier 10 and associated apparatus/systems of the present disclosure are configured to securely retain a coil spring C therein, and reliably and accurately allow extraction of the coil spring C. In order that the coil spring can be both securely retained and accurately extracted, the spring carrier 10 may be configured such that a certain clearance is provided between an outer diameter of the coil spring C and an inside wall of the inner cavity 13. The clearance is set to allow substantially unimpeded insertion and extraction of the coil spring C into/from the inner cavity 13, yet also minimise lateral play or movement of the coil spring C within the inner cavity so that the coil spring can be accurately discharged where required. In an embodiment, such clearance may be 0.05 mm-0.3 mmm, for instance between 0.1 mm-0.2 mm. In one embodiment, coil springs C to be received in the inner cavity 13 may have a maximum outer diameter of 9.95 mm. Accordingly, an internal diameter of the inner cavity 13 may be around 10.0 mm-12.95 mm, for instance around 10.05 mm-11.05 mm.

Although the embodiments of spring carrier 10 shown and described comprise two resilient arms 18, the invention is not intended to be limited to this configuration and in alternative embodiments, the spring carrier 10 may comprise only one, or more than two resilient arms 18. In embodiments comprising two or more resilient arms 18, the resilient arms 18 may be equally spaced around the perimeter of the spring carrier 10 for even and aligned retaining of a coil spring C in spring carrier 10. Additionally, such a configuration may also help promote the coil spring C being extracted evenly and in axial alignment with spring carrier 10 and into a component of medical device or manufacturing apparatus, for example, as intended.

The embodiments of spring carrier 10 shown and described comprise resilient arms 18 having one projecting element 25, and one notch 26, on each resilient arm 18. However, the invention is not intended to be limited to such a configuration and in alternative embodiments, a plurality of projecting elements 25 and/or a plurality of notches 26 may be provided on each resilient arm 18 configured such that the spring carrier may engage multiple turns of a coil spring C received within the hollow body 11. Such variants within the scope of the invention, optionally applicable to all embodiments described herein, may be as illustrated in FIGS. 12 to 14, for example.

The embodiments of spring carrier 10 shown and described comprise resilient arms 18 having an actuation feature which is engaged by an actuator 30 being inserted into the second opening 17 at the second end 15 of the hollow body 11. However, the invention is not intended to be limited to such a configuration, and in an alternative embodiment, the resilient arms 18 may include an actuation feature which extends outwardly of the hollow body 11. For example, the free ends 22 of the resilient arms 18 may project outwardly of the hollow body 11 and include an actuation feature. Such actuation feature may comprise a contact surface which may be disposed at an acute angle with respect to the central axis X-X of the hollow body 11. The actuator 30 may comprise a sleeve which may be located around the hollow body 11 and slid coaxially relative to the hollow body 11 to engage the actuation features of the resilient arms 18 to deflect the resilient arms 18 from the first position to the second position, to achieve the coil spring engagement/release function described above with the illustrated embodiments. Furthermore, as discussed above, the resilient arms may not include a specific actuation feature and may otherwise be manipulated in use to deflect and move as needed. For example, such alternative external actuator may effect mechanical engagement with the arm(s), such as by adhesion, vacuum contact, or other coupling.

The embodiments of spring carrier 10 shown and described comprise an opening 17 at the second end 15 of the hollow body 11. However, the invention is not intended to be limited to such a configuration, and in an alternative embodiment, second end 15 of the hollow body 11 may not comprise an opening. Such an arrangement may be present with the alternative configuration described above in which the actuation features of the resilient arms 18 extend outwardly of the hollow body 11 and are engaged by an actuator 30 externally of the hollow body 11.

Some of the embodiments of spring carrier 10 shown and described comprise an opening 17 at the second end 15 of the hollow body 11 which is of the same size and dimensions as the cross-sectional dimension of the inner cavity 13 of the hollow body 11. However, the invention is not intended to be limited to such a configuration, and in an alternative embodiment, the second end 15 of the hollow body 11 may include an opening which is of a smaller cross-sectional dimension, and/or a different shape, than the cross-section of the inner cavity 13. Such an alternative configuration may still permit use of an actuator 30 which engages and moves the resilient arms 18 via insertion through the opening 17 at the second end 15, or may be used in combination with the externally-accessible actuation features of the resilient arms 18 described above.

Some embodiments disclosed herein comprise a continuous annular portion 27 extending entirely around the perimeter of the hollow body 11. Such feature may optionally be applicable to all embodiments described herein. However, the invention is not intended to be limited to such features and embodiments envisaged within the scope of the invention may not comprise such feature.

Some embodiments disclosed herein comprise a flange 28 extending around the perimeter of the hollow body 11 at the first proximal end 14 thereof. Such feature may optionally be applicable to all embodiments described herein. However, the invention is not intended to be limited to such feature and embodiments envisaged within the scope of the invention may not comprise a flange 28, or may comprise a flange disposed along the length of the hollow body other than at the remote end of the first proximal end, for example, at the second distal end 15, or intermediate the first proximal end and the second distal end.

Embodiments of spring carrier 10 described herein comprise at least one deflectable member configured to engage and retain a coil spring within the inner cavity 13 of the hollow body 11. The at least one deflectable member is provided proximate to an end of the hollow body opposite to that into and from which a coil spring is inserted/extracted in use. This arrangement may help avoid interference between the coil spring and a spring retaining/actuating mechanism, since the spring is inserted/extracted at one end and the actuation of the deflectable member occurs at the opposite end. This may help towards providing a simple and reliable manufacturing/assembly apparatus and process. Furthermore, in the exemplary embodiments illustrated and described, the engagement of the deflectable member(s) and/or retaining formation(s) with the coil spring to retain the coil spring, is effected by direct contact between the deflectable member(s) and/or retaining formation(s) with the coil spring

Those of skill in the art will understand that modifications (additions and/or removals) of various components of the devices, apparatuses, methods, and embodiments described herein may be made without departing from the full scope and spirit of the present invention, which encompass such modifications and any and all equivalents thereof.

1. A spring carrier (10) for receiving, retaining and discharging of a coil spring in a manufacturing assembly process comprising:
an elongate hollow body (11) defining an inner cavity (13) configured to receive a coil spring (C);
an opening (16) at a first proximal end (14) of the hollow body for the insertion and/or extraction of a coil spring into/from the inner cavity;
the hollow body including a second distal end (15) opposite to the first proximal end;
at least one deflectable member (18) located proximate to the second distal end of the hollow body and including a retaining formation (25, 26) which is configured to engage and retain a coil spring when located within the inner cavity;
wherein the deflectable member is movable between a first unbiased position, whereby the retaining formation extends into the inner cavity to engage and retain the coil spring when located within the inner cavity, and a second biased position whereby the retaining formation is disposed outwardly to disengage a coil spring when located within the inner cavity.

2. A spring carrier (10) according to claim 1, wherein the deflectable member (18) extends substantially parallel to a central axis (X-X) of the hollow body (11) in the first position.

3. A spring carrier (10) according to claim 1 or claim 2, wherein the deflectable member (18) includes an actuation feature (23) for engagement with an actuator (30) to deflect the deflectable member from the first position to the second position.

4. A spring carrier (10) according to any preceding claim wherein the deflectable member (18) includes an abutment step (34) for engagement with an end of a coil spring (C) when retained within the inner cavity (13).

5. A spring carrier (10) according to any preceding claim, wherein the retaining formation comprises at least one projecting element (25) extending inwardly from the deflectable member (18).

6. A spring carrier (10) according to any preceding claim wherein the hollow body (11) comprises a continuous annular portion (27) extending entirely around the perimeter of the hollow body at a distal-most region of the second end (15) and located further towards the second end than the deflectable member (18).

7. A spring carrier (10) according to any preceding claim wherein the hollow body (11) is a cylindrical tube which is circular in cross-section.

8. A spring carrier (10) according to any preceding claim, wherein the hollow body (11) includes a flange (28) extending radially outwardly from the hollow body.

9. A spring carrier (10) according to any preceding claim, comprising an opening (17) at the second end (15) of the hollow body (11).

10. A spring carrier (10) according to any preceding claim comprising at least one window (60) in at least one of a side wall of the hollow body and the deflectable member to allow a coil spring (C) located within the spring carrier to be visible from outside the spring carrier through the window.

11. A spring carrier (10) according to any preceding claim where the first opening (16) at the first end (14) of the hollow body (11) comprises a tapered region (16A) such that the first opening widens towards the first proximal end.

12. An apparatus comprising:
a spring carrier (10) according to any preceding claim; and
an actuator (30) configured for engagement with the deflectable member (18) and operable to move the deflectable member from the first position to the second position.

13. An apparatus according to claim 12, wherein the actuator (30) comprises an elongate rod configured to be inserted into an opening (17) at the second distal end (15) of the hollow body (11), and optionally wherein the actuator (30) comprises a chamfered end (33) configured to engage with the deflectable member (18).

14. A method of manipulating a coil spring (C) using a spring carrier (10) for receiving, retaining and discharging of a coil spring in a manufacturing assembly process, the spring carrier comprising an elongate hollow body (11) defining an inner cavity (13), an opening (16) at a first proximal end (14) of the hollow body, a second distal end (15) opposite to the first proximal end, and at least one deflectable member (18) located proximate to the second distal end of the hollow body and including a retaining formation (25, 26), the method comprising moving the deflectable member (18) from a first position whereby the retaining formation extends into the inner cavity, to a second position whereby the retaining formation extends outwardly, inserting the coil spring into the inner cavity (13) through the opening (16) at the first end (14) of the hollow body (11), and moving the deflectable member from the second position to the first position such that the retaining formation (25, 26) engages the coil spring to retain the coil spring within the inner cavity

15. The method of claim 14, wherein the method comprises engaging an actuator (30) with the deflectable member (18) to move the deflectable member from the first position to the second position, and disengaging the actuator after insertion of the coil spring (C) into the inner cavity (13) to allow the deflectable member to move to the first position such that the retaining formation engages the coil spring to retain the coil spring within the inner cavity.
